SEQUENCE LISTING (78 sequences)

SEQ ID NO: 1 - protein, 118 aa, unidentified
  source 1..118: note = rabbit
QELEESGGDLVKPGASLTLTCTASGFFFSAENHICWVRQAPGKGLEWIGCVYIGSYVGGSGDPYYASWAKGRFTISKTSSTTVTLQMSSLTAADTATYFCARDLGSLWGPGTLVTVSS

SEQ ID NO: 2 - protein, 110 aa, unidentified
  source 1..110: note = rabbit
DVVMTQTASSVSAAVGGTVTIACQSSQSVYDNNNLAWFQQKPGQPPKRLIYSASTLASGVSSRFKGSGYGTQFTLTISDVQCDDAATYYCVGAFSGYIFVFGGGTEVVVK

SEQ ID NO: 3 - protein, 11 aa, unidentified
  source 1..11: note = rabbit
GFFFSAENHIC

SEQ ID NO: 4 - protein, 16 aa, unidentified
  source 1..16: note = rabbit
CVYIGSYVGGSGDPYY

SEQ ID NO: 5 - protein, 5 aa, unidentified
  source 1..5: note = rabbit
DLGSL

SEQ ID NO: 6 - protein, 13 aa, unidentified
  source 1..13: note = rabbit
QSSQSVYDNNNLA

SEQ ID NO: 7 - protein, 7 aa, unidentified
  source 1..7: note = rabbit
SASTLAS

SEQ ID NO: 8 - protein, 10 aa, unidentified
  source 1..10: note = rabbit
VGAFSGYIFV

SEQ ID NO: 9 - protein, 10 aa, unidentified
  source 1..10: note = rabbit
FFFSAENHIC

SEQ ID NO: 10 - protein, 19 aa, unidentified
  source 1..19: note = rabbit
WIGCVYIGSYVGGSGDPYY

SEQ ID NO: 11 - protein, 6 aa, unidentified
  source 1..6: note = rabbit
RDLGSL

SEQ ID NO: 12 - protein, 10 aa, unidentified
  source 1..10: note = rabbit
QSVYDNNNLA

SEQ ID NO: 13 - protein, 11 aa, unidentified
  source 1..11: note = rabbit
RLIYSASTLAS

SEQ ID NO: 14 - protein, 10 aa, unidentified
  source 1..10: note = rabbit
VGAFSGYIFV

SEQ ID NO: 15 - protein, 28 aa, synthetic construct
  source 1..28: note = synthetic peptide for antibody production
CQRQMSEPNIPFPPQGFKQEYHDPVYEH

SEQ ID NO: 16 - protein, 14 aa, Homo sapiens
MSEPNIPFPPQGFK

SEQ ID NO: 17 - protein, 476 aa, Homo sapiens
MDGFYDQQVPYMVTNSQRGRNCNEKPTNVRKRKFINRDLAHDSEELFQDLSQLQETWLAEAQVPDNDEQFVPDYQAESLAFHGLPLKIKKEPHSPCSEISSACSQEQPFKFSYGEKCLYNVSAYDQKPQVGMRPSNPPTPSSTPVSPLHHASPNSTHTPKPDRAFPAHLPPSQSIPDSSYPMDHRFRRQLSEPCNSFPPLPTMPREGRPMYQRQMSEPNIPFPPQGFKQEYHDPVYEHNTMVGSAASQSFPPPLMIKQEPRDFAYDSEVPSCHSIYMRQEGFLAHPSRTEGCMFEKGPRQFYDDTCVVPEKFDGDIKQEPGMYREGPTYQRRGSLQLWQFLVALLDDPSNSHFIAWTGRGMEFKLIEPEEVARRWGIQKNRPAMNYDKLSRSLRYYYEKGIMQKVAGEYVYKFVCDPEALFSMAFPDNQRPLLKTDMERHINEEDTVPLSHFDESMAYMPEGGCCNPHPYNEGYVY

SEQ ID NO: 18 - protein, 18 aa, Homo sapiens
MSEPNIPFPPQGFKQEYH

SEQ ID NO: 19 - protein, 27 aa, synthetic construct
  source 1..27: note = synthetic peptide used as assay for antibodies
QRQMSEPNIPFPPQGFKQEYHDPVYEH

SEQ ID NO: 20 - protein, 12 aa, synthetic construct
  source 1..12: note = synthetic peptide used as assay for antibodies
QRQMSEPNIPFP

SEQ ID NO: 21 - protein, 12 aa, synthetic construct
  source 1..12: note = synthetic peptide used as assay for antibodies
MSEPNIPFPPQG

SEQ ID NO: 22 - protein, 12 aa, synthetic construct
  source 1..12: note = synthetic peptide used as assay for antibodies
PNIPFPPQGFKQ

SEQ ID NO: 23 - protein, 12 aa, synthetic construct
  source 1..12: note = synthetic peptide used as assay for antibodies
PFPPQGFKQEYH

SEQ ID NO: 24 - protein, 12 aa, synthetic construct
  source 1..12: note = synthetic peptide used as assay for antibodies
PQGFKQEYHDPV

SEQ ID NO: 25 - protein, 12 aa, synthetic construct
  source 1..12: note = synthetic peptide used as assay for antibodies
FKQEYHDPVYEH

SEQ ID NO: 26 - protein, 26 aa, synthetic construct
  source 1..26: note = synthetic peptide used as assay for antibodies
RQMSEPNIPFPPQGFKQEYHDPVYEH

SEQ ID NO: 27 - protein, 25 aa, synthetic construct
  source 1..25: note = synthetic peptide used as assay for antibodies
QMSEPNIPFPPQGFKQEYHDPVYEH

SEQ ID NO: 28 - protein, 24 aa, synthetic construct
  source 1..24: note = synthetic peptide used as assay for antibodies
MSEPNIPFPPQGFKQEYHDPVYEH

SEQ ID NO: 29 - protein, 23 aa, synthetic construct
  source 1..23: note = synthetic peptide used as assay for antibodies
SEPNIPFPPQGFKQEYHDPVYEH

SEQ ID NO: 30 - skipped

SEQ ID NO: 31 - protein, 20 aa, synthetic construct
  source 1..20: note = synthetic peptide used as assay for antibodies
NIPFPPQGFKQEYHDPVYEH

SEQ ID NO: 32 - protein, 19 aa, synthetic construct
  source 1..19: note = synthetic peptide used as assay for antibodies
IPFPPQGFKQEYHDPVYEH

SEQ ID NO: 33 - protein, 18 aa, synthetic construct
  source 1..18: note = synthetic peptide used as assay for antibodies
PFPPQGFKQEYHDPVYEH

SEQ ID NO: 34 - protein, 17 aa, synthetic construct
  source 1..17: note = synthetic peptide used as assay for antibodies
FPPQGFKQEYHDPVYEH

SEQ ID NO: 35 - protein, 14 aa, synthetic construct
  source 1..14: note = synthetic peptide used as assay for antibodies
PQGFKQEYHDPVYE

SEQ ID NO: 36 - protein, 14 aa, synthetic construct
  source 1..14: note = synthetic peptide used as assay for antibodies
QGFKQEYHDPVYEH

SEQ ID NO: 37 - protein, 13 aa, synthetic construct
  source 1..13: note = synthetic peptide used as assay for antibodies
GFKQEYHDPVYEH

SEQ ID NO: 38 - protein, 12 aa, synthetic construct
  source 1..12: note = synthetic peptide used as assay for antibodies
QRQMSEPNIPFP

SEQ ID NO: 39 - protein, 13 aa, synthetic construct
  source 1..13: note = synthetic peptide used as assay for antibodies
QRQMSEPNIPFPP

SEQ ID NO: 40 - protein, 14 aa, synthetic construct
  source 1..14: note = synthetic peptide used as assay for antibodies
QRQMSEPNIPFPPQ

SEQ ID NO: 41 - protein, 15 aa, synthetic construct
  source 1..15: note = synthetic peptide used as assay for antibodies
QRQMSEPNIPFPPQG

SEQ ID NO: 42 - protein, 17 aa, synthetic construct
  source 1..17: note = synthetic peptide used as assay for antibodies
QRQMSEPNIPFPPQGFK

SEQ ID NO: 43 - protein, 18 aa, synthetic construct
  source 1..18: note = synthetic peptide used as assay for antibodies
QRQMSEPNIPFPPQGFKQ

SEQ ID NO: 44 - protein, 19 aa, synthetic construct
  source 1..19: note = synthetic peptide used as assay for antibodies
QRQMSEPNIPFPPQGFKQE

SEQ ID NO: 45 - protein, 20 aa, synthetic construct
  source 1..20: note = synthetic peptide used as assay for antibodies
QRQMSEPNIPFPPQGFKQEY

SEQ ID NO: 46 - protein, 21 aa, synthetic construct
  source 1..21: note = synthetic peptide used as assay for antibodies
QRQMSEPNIPFPPQGFKQEYH

SEQ ID NO: 47 - protein, 22 aa, synthetic construct
  source 1..22: note = synthetic peptide used as assay for antibodies
QRQMSEPNIPFPPQGFKQEYHD

SEQ ID NO: 48 - protein, 23 aa, synthetic construct
  source 1..23: note = synthetic peptide used as assay for antibodies
QRQMSEPNIPFPPQGFKQEYHDP

SEQ ID NO: 49 - protein, 24 aa, synthetic construct
  source 1..24: note = synthetic peptide used as assay for antibodies
QRQMSEPNIPFPPQGFKQEYHDPV

SEQ ID NO: 50 - protein, 25 aa, synthetic construct
  source 1..25: note = synthetic peptide used as assay for antibodies
QRQMSEPNIPFPPQGFKQEYHDPVY

SEQ ID NO: 51 - protein, 26 aa, synthetic construct
  source 1..26: note = synthetic peptide used as assay for antibodies
QRQMSEPNIPFPPQGFKQEYHDPVYE

SEQ ID NO: 52 - protein, 27 aa, synthetic construct
  source 1..27: note = synthetic peptide used as assay for antibodies
ARQMSEPNIPFPPQGFKQEYHDPVYEH

SEQ ID NO: 53 - protein, 27 aa, synthetic construct
  source 1..27: note = synthetic peptide used as assay for antibodies
QAQMSEPNIPFPPQGFKQEYHDPVYEH

SEQ ID NO: 54 - protein, 27 aa, synthetic construct
  source 1..27: note = synthetic peptide used as assay for antibodies
QRAMSEPNIPFPPQGFKQEYHDPVYEH

SEQ ID NO: 55 - protein, 27 aa, synthetic construct
  source 1..27: note = synthetic peptide used as assay for antibodies
QRQASEPNIPFPPQGFKQEYHDPVYEH

SEQ ID NO: 56 - protein, 27 aa, synthetic construct
  source 1..27: note = synthetic peptide used as assay for antibodies
QRQMAEPNIPFPPQGFKQEYHDPVYEH

SEQ ID NO: 57 - protein, 27 aa, synthetic construct
  source 1..27: note = synthetic peptide used as assay for antibodies
QRQMSAPNIPFPPQGFKQEYHDPVYEH

SEQ ID NO: 58 - protein, 27 aa, synthetic construct
  source 1..27: note = synthetic peptide used as assay for antibodies
QRQMSEANIPFPPQGFKQEYHDPVYEH

SEQ ID NO: 59 - protein, 27 aa, synthetic construct
  source 1..27: note = synthetic peptide used as assay for antibodies
QRQMSEPAIPFPPQGFKQEYHDPVYEH

SEQ ID NO: 60 - protein, 27 aa, synthetic construct
  source 1..27: note = synthetic peptide used as assay for antibodies
QRQMSEPNAPFPPQGFKQEYHDPVYEH

SEQ ID NO: 61 - protein, 27 aa, synthetic construct
  source 1..27: note = synthetic peptide used as assay for antibodies
QRQMSEPNIAFPPQGFKQEYHDPVYEH

SEQ ID NO: 62 - protein, 27 aa, synthetic construct
  source 1..27: note = synthetic peptide used as assay for antibodies
QRQMSEPNIPAPPQGFKQEYHDPVYEH

SEQ ID NO: 63 - protein, 27 aa, synthetic construct
  source 1..27: note = synthetic peptide used as assay for antibodies
QRQMSEPNIPFAPQGFKQEYHDPVYEH

SEQ ID NO: 64 - protein, 27 aa, synthetic construct
  source 1..27: note = synthetic peptide used as assay for antibodies
QRQMSEPNIPFPAQGFKQEYHDPVYEH

SEQ ID NO: 65 - protein, 27 aa, synthetic construct
  source 1..27: note = synthetic peptide used as assay for antibodies
QRQMSEPNIPFPPAGFKQEYHDPVYEH

SEQ ID NO: 66 - protein, 27 aa, synthetic construct
  source 1..27: note = synthetic peptide used as assay for antibodies
QRQMSEPNIPFPPQAFKQEYHDPVYEH

SEQ ID NO: 67 - protein, 27 aa, synthetic construct
  source 1..27: note = synthetic peptide used as assay for antibodies
QRQMSEPNIPFPPQGAKQEYHDPVYEH

SEQ ID NO: 68 - protein, 27 aa, synthetic construct
  source 1..27: note = synthetic peptide used as assay for antibodies
QRQMSEPNIPFPPQGFAQEYHDPVYEH

SEQ ID NO: 69 - protein, 27 aa, synthetic construct
  source 1..27: note = synthetic peptide used as assay for antibodies
QRQMSEPNIPFPPQGFKAEYHDPVYEH

SEQ ID NO: 70 - protein, 27 aa, synthetic construct
  source 1..27: note = synthetic peptide used as assay for antibodies
QRQMSEPNIPFPPQGFKQAYHDPVYEH

SEQ ID NO: 71 - protein, 27 aa, synthetic construct
  source 1..27: note = synthetic peptide used as assay for antibodies
QRQMSEPNIPFPPQGFKQEAHDPVYEH

SEQ ID NO: 72 - protein, 27 aa, synthetic construct
  source 1..27: note = synthetic peptide used as assay for antibodies
QRQMSEPNIPFPPQGFKQEYADPVYEH

SEQ ID NO: 73 - protein, 27 aa, synthetic construct
  source 1..27: note = synthetic peptide used as assay for antibodies
QRQMSEPNIPFPPQGFKQEYHAPVYEH

SEQ ID NO: 74 - protein, 27 aa, synthetic construct
  source 1..27: note = synthetic peptide used as assay for antibodies
QRQMSEPNIPFPPQGFKQEYHDAVYEH

SEQ ID NO: 75 - protein, 27 aa, synthetic construct
  source 1..27: note = synthetic peptide used as assay for antibodies
QRQMSEPNIPFPPQGFKQEYHDPAYEH

SEQ ID NO: 76 - protein, 27 aa, synthetic construct
  source 1..27: note = synthetic peptide used as assay for antibodies
QRQMSEPNIPFPPQGFKQEYHDPVAEH

SEQ ID NO: 77 - protein, 27 aa, synthetic construct
  source 1..27: note = synthetic peptide used as assay for antibodies
QRQMSEPNIPFPPQGFKQEYHDPVYAH

SEQ ID NO: 78 - protein, 27 aa, synthetic construct
  source 1..27: note = synthetic peptide used as assay for antibodies
QRQMSEPNIPFPPQGFKQEYHDPVYEA

(19) United States
(12) Patent Application Publication
(10) Pub. No.: US 2026/0227403 A1
(43) Pub. Date: Aug. 6, 2026

(54) ETV1 MONOCLONAL ANTIBODIES

(71) Applicant: THE HENRY M. JACKSON FOUNDATION FOR THE ADVANCEMENT OF MILITARY MEDICINE, INC., Bethesda, MD (US)

(72) Inventors: Shyh-Han TAN, Bethesda, MD (US); Albert DOBI, Bethesda, MD (US); Shiv K. SRIVASTAVA, Bethesda, MD (US)

(21) Appl. No.: 19/149,189

(22) PCT Filed: Jan. 18, 2024
(86) PCT No.: PCT/US2024/012006

§ 371 Date: Jul. 18, 2025

Related U.S. Application Data
(60) Provisional application No. 63/439,905, filed on Jan. 19, 2023.

(51) Int. Cl. G01N 33/575; C07K 16/30

(52) U.S. Cl. CPC G01N 33/575; C07K 16/30; C07K 2317/33; C07K 2317/34; C07K 2317/565; C07K 2317/92; G01N 2800/7028

(57) ABSTRACT

Disclosed are monoclonal antibodies, antigen-binding fragments thereof, and other binding molecules derived therefrom that bind to ETV1 (ETS Variant Transcription Factor 1). Also disclosed are epitopes of ETV1 that can be used to make antibodies that bind to ETV1. In addition, methods of using these antibodies, or antigen-binding fragments thereof, are disclosed, including methods of detecting ETV1 in a sample, and methods of diagnosing and treating cancer.

### CROSS-REFERENCE TO RELATED APPLICATIONS

This application claims priority to U.S. Provisional Application No. 63/439,905 filed 19 Jan. 2023, the entire contents of which are hereby incorporated by reference in their entirety.

### ACKNOWLEDGMENT OF GOVERNMENT SUPPORT

This invention was made with government support under ACN12011-001 and ACN17005-001 awarded by the National Institutes of Health and under HU0001-21-2-0021 awarded by the Uniformed Services University of the Health Sciences. The government has certain rights in the invention.

### SEQUENCE LISTING

The Sequence Listing associated with this application is filed in electronic format as an XML file and hereby incorporated by reference into the specification in its entirety. The name of the XML file containing the Sequence Listing is HMJ_186_PCT_SL.xml and the size of the text file is 84,156 bytes.

### FIELD

This application relates generally to monoclonal antibodies, antigen-binding fragments thereof, and other binding molecules derived therefrom that bind to ETV1 (ETS Variant Transcription Factor 1). It also discloses the use of these antibodies, antigen-binding fragments thereof, and binding molecules derived therefrom.

### BACKGROUND

The ETS family of transcription factors, characterized by the presence of a conserved DNA-binding domain (ETS-domain), are encoded by 28 genes in the human genome. While most ETS factors are ubiquitously expressed in almost all cell types, they are frequently involved in genomic fusions and translocation events that lead to their overexpression in multiple cancers. For example, SPDEF (SAM pointed domain-containing ETS transcription factor, PDEF), ELK4 (ETS domain-containing protein ELK-4, SAP1), ELF3 (ETS-related transcription factor ELF-3/ESE1) and EHF (ETS homologous factor/ESE3) are expressed in the normal prostate (5-7), whereas ERG (ETS Transcription Factor ERG), ETV1 (ETS translocation variant 1/ER81), ETV4 (ETS translocation variant 4/E1AF/PEA3), ETV5 (ETS translocation variant 5/ERM), and FLI1 (Friend leukemia integration 1 transcription factor/EWSR2) are only overexpressed because of chromosomal rearrangement events. In primary prostate cancers, the fusion of TMPRSS2 to the ERG gene is the most frequent alteration of the ETS gene fusion subtypes. TMPRSS2 also fuses to other ETS family genes such as ETV1, ETV4 and ETV5 in a smaller subset of prostate cancers but these ETS genes also form rearrangements with other 5′-partners. For example, SLC45A3, C15orf21, and HNRPA2B1, form the 5′-gene fusion partner that respond differently to androgen. Altogether, ETV1 and ETV4 gene fusions occur in approximately 5 to 10% of cases, they are almost always mutually exclusive from TMPRSS2-ERG fusion or PTEN deletion and are correlated with poor outcome.

In other tissues, ETV1, together with ETV4, and ETV5 are transcriptionally repressed by CIC, a high-mobility group (HMG)-box protein encoded by the transcriptional repressor Capicua (CIC) gene. Loss-of-function CIC mutations in tumors, such as oligodendrogliomas, and gene fusions of CIC with either DUX4 or FOXO4 in round cell sarcomas result in upregulated ETV1 and ETV4 overexpression. Analysis of microarray gene expression profiles of gliomas revealed sustained ETV1 and ETV4 expression that increases with glioma grade. ETV1 transcriptional expression is also activated by upstream mutations of KIT that encode constitutively active KIT tyrosine kinase receptor. In the mouse brain, KIT and ETV1 were found to co-express in regions where germinomas, a subtype of central nervous system germ-cell tumor, develop. Since cases of germinomas in humans also coincide with regions of ETV) and KIT co-expression, they were thought to share a similar mechanism with other KIT-driven cancers that arise from a brain-cell progenitor. Gastrointestinal stromal tumors (GIST) are the most common mesenchymal tumors that occur in the stomach (60%), small intestine (30%), but also elsewhere in the GI-tract and the intra-abdominal soft tissues. In interstitial cells of Cajal (ICC), the presumed origin of GIST, the activation of the KIT pathway triggers both high expression of ETV1 and prolongs ETV1 protein stability to promote tumorigenesis. ETV1 synergistically cooperates with KIT as a lineage survival factor to mediate transformation by transcriptionally activating many of the known GIST biomarkers, such as H19 lincRNA, which has been shown to act both as tumor suppressor and oncogene in different types of cancer.

The frequent overexpression of ETV1 in multiple malignancies underscores its utility as a potential diagnostic marker and therapeutic target. Nevertheless, our understanding of the oncogenic function of ETV1 in various cancers and in-depth knowledge of the association between ETV1 expression and clinical or pathologic features of various cancer types are prevented by the lack of specific antibodies against ETV1. Specifically, attempts to raise highly specific and high affinity mouse monoclonal antibodies against ETV1 in mice have been unsuccessful, likely because the protein is highly conserved between human and mouse.

There is a need for reagents, in particular monoclonal antibodies, and methods that permit detection of ETV1 and/or treatment of diseases, disorders, and conditions associated with ETV1 expression.

### SUMMARY

Disclosed herein are monoclonal antibodies that binds to an epitope of ETV1 (ETS Variant Transcription Factor 1), or antigen-binding fragments thereof. These monoclonal antibodies and antigen-binding fragments thereof specifically bind to ETV1 and are able to distinguish ETV1 from ETV5, ETV4 and other ETS factors. Because of their binding specificity, these monoclonal antibodies and antigen-binding fragments thereof can be used to diagnose, or confirm the diagnosis of, cancers that overexpress ETV1, including but not limited to prostate cancer, gastrointestinal stromal tumors, small round cell sarcoma, melanoma, and gliomas, as well as for treating those cancers. These monoclonal antibodies and antigen-binding fragments thereof can also be used as a reagent for research to detect ETV1 and study the biological function of ETV1 in comparison to other ubiquitous ETS transcription factors. These monoclonal antibodies and antigen-binding fragments thereof can be conjugated with tracer chemicals for use in clinical imaging, conjugated with drugs to deliver therapeutic payloads to ETV1 expressing cancer cells, or combined with subunits of other specific antibodies to create bispecific antibodies.

In one aspect, this application describes the isolation and characterization of monoclonal antibodies and antigen-binding fragments thereof that specifically bind to an epitope of ETV1. In some embodiments, the monoclonal antibodies and antigen-binding fragments thereof of the disclosure comprise a heavy chain variable domain comprising a complementarity determining region 1 (CDR1) comprising the amino acid sequence of SEQ ID NO: 3, a CDR2 comprising the amino acid sequence of SEQ ID NO: 4, and a CDR3 comprising the amino acid sequence of SEQ ID NO: 5, and a light chain variable domain comprising a CDR1 comprising the amino acid sequence of SEQ ID NO: 6, a CDR2 comprising the amino acid sequence of SEQ ID NO: 7, and a CDR3 comprising the amino acid sequence of SEQ ID NO: 8. In some embodiments, the monoclonal antibodies and antigen-binding fragments thereof of the disclosure comprise a heavy chain variable domain comprising a CDR1 comprising the amino acid sequence of SEQ ID NO: 9, a CDR2 comprising the amino acid sequence of SEQ ID NO: 10, and a CDR3 comprising the amino acid sequence of SEQ ID NO: 11, and a light chain variable domain comprising a CDR1 comprising the amino acid sequence of SEQ ID NO: 12, a CDR2 comprising the amino acid sequence of SEQ ID NO: 13, and a CDR3 comprising the amino acid sequence of SEQ ID NO: 14. In some embodiments, the monoclonal antibodies and antigen-binding fragments thereof of the disclosure comprise a heavy chain variable domain comprising an amino acid sequence having at least 90% sequence identity to SEQ ID NO: 1, preferably the amino acid sequence of SEQ ID NO: 1, and a light chain variable domain comprising an amino acid sequence having at least 90% sequence identity to SEQ ID NO: 2, preferably the amino acid sequence of SEQ ID NO: 2. In some embodiments, the monoclonal antibodies and antigen-binding fragments thereof of the disclosure are rabbit monoclonal antibodies. In some embodiments, the monoclonal antibodies and antigen-binding fragments thereof of the disclosure bind to an epitope of ETV1 comprising the amino acid sequence of SEQ ID NO: 15. In some embodiments, the monoclonal antibodies and antigen-binding fragments thereof of the disclosure bind to an epitope of ETV1 comprising the amino acid sequence of SEQ ID NO: 16. A pharmaceutical composition comprising the monoclonal antibodies and antigen-binding fragments thereof of the disclosure and a pharmaceutically acceptable excipient, as well as a nucleic acid molecule encoding the monoclonal antibodies and antigen-binding fragments thereof of the disclosure and a host cell comprising the nucleic acid molecule, are also disclosed. Also provided is a kit for use in detecting ETV1 expression comprising the monoclonal antibodies and antigen-binding fragments thereof of the disclosure in a container, a control, and instructions for carrying out an immunoassay for determination of ETV1 expression.

In another aspect, the disclosure provides methods of using the monoclonal antibodies and antigen-binding fragments thereof disclosed herein. In some embodiments, provided are methods of determining the likelihood of a subject having a cancer, the method comprising determining a level of ETV1 expression in a sample obtained from the subject using the monoclonal antibodies and antigen-binding fragments thereof disclosed herein and comparing the level of ETV1 expression with a reference level of ETV1 expression. In such embodiments, if the reference level of ETV1 expression is obtained from a subject known not to have a cancer, an increased level of ETV1 expression in the sample as compared to the reference level of ETV1 expression indicates that there is an increased likelihood that the subject has a cancer. Alternatively, if the reference level of ETV1 expression is obtained from a subject known to have a cancer, a decreased level of ETV1 expression in the sample as compared to the reference level of ETV1 expression indicates that there is an increased likelihood that the subject does not have a cancer. In other embodiments, provided are methods of diagnosing a cancer associated with overexpression of ETV1 in a subject in need thereof, the method comprising determining a level of ETV1 expression in a sample obtained from the subject using the monoclonal antibodies and antigen-binding fragments thereof disclosed herein and comparing the level of ETV1 expression with a reference level of ETV1 expression. In such embodiments, if the reference level of ETV1 expression is obtained from a subject known not to have a cancer, an increased level of ETV1 expression in the sample as compared to the reference level of ETV1 expression indicates that there is an increased likelihood that the subject has a cancer, or if the reference level of ETV1 expression is obtained from a subject known to have a cancer associated with overexpression of ETV1, a decreased level of ETV1 expression in the sample as compared to the reference level of ETV1 expression indicates that there is an increased likelihood that the subject does not have a cancer. In some embodiments, the level of ETV1 expression in the sample is determined using an analysis selected from the group consisting of Western blotting, protein gel electrophoresis, immunoprecipitation, ELISA, immunohistochemistry, and combinations thereof. In some embodiments, the level of ETV1 expression in the sample is determined by performing immunohistochemistry on the sample. In some embodiments, the subject is a human suspected of having a cancer, particularly a cancer associated with overexpression of ETV1. In some embodiments, the cancer is prostate cancer, gastrointestinal stromal tumor, small round cell sarcoma, melanoma, or gliomas.

Also provided are methods of treating a cancer associated with overexpression of ETV1, the method comprising administering to a subject in need thereof a therapeutically effective amount of the monoclonal antibodies and antigen-binding fragments thereof disclosed herein, or a pharmaceutical composition comprising the same. The cancer associated with overexpression of ETV1, in some embodiments, is prostate cancer, gastrointestinal stromal tumor, small round cell sarcoma, melanoma, or gliomas.

Methods of detecting ETV1 expression in a sample are also provided. Such methods comprise, in some embodiments, incubating the sample with the monoclonal antibodies or the antigen-binding fragments thereof disclosed herein under conditions permitting the detection of ETV1 expression when ETV1 is present in the sample. In some embodiments, the ETV1 expression is detected using an analysis selected from the group consisting of Western blotting, protein gel electrophoresis, immunoprecipitation, ELISA, immunohistochemistry, and combinations thereof. In some embodiments, the sample is a biological sample comprising cells.

In a further aspect, provided herein are immunogenic compositions comprising an epitope of ETV1, wherein the epitope of ETV1 comprises the amino acid sequence of SEQ ID NO: 15 or SEQ ID NO: 16. In some embodiments, the epitope comprises the amino acid of SEQ ID NO: 16 and contains no more than 30 amino acids. In some embodiments, the epitope comprises the amino acid sequence of SEQ ID NO: 15 and contains no more than 15 amino acids. In some embodiments, the immunogenic compositions disclose herein further comprise an adjuvant.

The immunogenic compositions disclosed herein can be used to generate antibodies, particularly monoclonal antibodies, that binds to ETV. Accordingly, in some embodiments, provided herein is a method of generating a monoclonal antibody that binds to ETV1, the method comprising administering the immunogenic composition disclosed herein to a non-human mammal, isolating B cells from the non-human mammal, immortalizing the B cells to create a cell line capable of producing a monoclonal antibody, and selecting the monoclonal antibody that binds to the epitope of ETV1 comprising the amino acid sequence of SEQ ID NO: 15 or SEQ ID NO: 16.

### BRIEF DESCRIPTION OF THE DRAWINGS

FIG. 1 shows that ETV1 rabbit mAb (29E4) binds ETV1 selectively and does not cross-react against ETV4, ETV5, ERG, FLI and SPDEF. Immunoblots using antibodies against ETV1 (A and H), ETV4 (B), ERG (C), SPDEF (D), FLI1 (E), FLAG (F), and GAPDH (G), as a loading control. Panels A to G show immunoblots loaded with cell lysates from PC3 (lanes 1-2 at 50 μg per lane), HEK-293 (lanes 3-9 at 2 μg per lane) and LNCaP cells (lanes 9-10 at 2 μg per lane). PC3 cells were transfected with 25 nM non-targeting (NT) siRNA or ETV-1 siRNA oligos (lanes 1 and 2, respectively). HEK-293 cell lysates were transfected with either an empty plasmid (lane 4) or expression vector encoding ETV1 (lane 3) or ERG (lane 8), FLAG-tagged ETV1 (lane 5), ETV4 (lane 6), ETV5 (lane 7), or SPDEF (lane 9). LNCaP cell lysates were infected with either a control vector (lane 10) or adenoviral vector expressing FLI1 (lanes 11). Panel H shows a longer exposure of an immunoblot of PC3 cell lysates (50 μg per lane) transfected with 25 nM NT siRNA or ETV-1 siRNA oligos at 25 nM or 50 nM concentrations.

FIG. 2 shows that nuclear localized ETV1 protein in PC3 cells is detected by ETV1 29E4 mAb. Immunofluorescence assay of PC3 cells stained with ETV1 mAb followed by Alexa-Fluor-594 goat anti-rabbit secondary antibody to show ETV1 expression. Cells were transfected with 25 nM and 50 nM of control NT siRNA or ETV1 specific siRNA oligonucleotides. Cellular actin and nuclear DNA were labelled with Alexa-488 labelled phalloidin and DAPI staining, respectively.

FIG. 3 shows identification of minimal epitopes recognized by ETV1 29E4 mAb by ELISA using overlapping, truncated, and alanine substituted peptides. (A) The immunogenic peptide, P1 (SEQ ID NO: 19), was used as a positive control. Peptides P2 to P7 contain 12 amino acids with an overlap of 9 residues between each peptide (P2: SEQ ID NO: 20; P3: SEQ ID NO: 21; P4: SEQ ID NO: 22; P5: SEQ ID NO: 23; P6: SEQ ID NO: 24; P7: SEQ ID NO: 25). (B) Line plots showing the average absorbance from duplicate assays quantitating the binding of 500 ng per well of each overlapping peptide to ETV1 mAb diluted at 0, 0.25, 1, 4, 15, 60, 250 to 1000 ng/mL concentrations. (C) Amino acid sequence of peptides truncated from both the N- and C-termini of immunogenic peptide P1 (SEQ ID NO: 19). Residues bounded by the box and highlighted in the wild-type sequence at the bottom (SEQ ID NO: 19) indicate the minimal epitope recognized by the ETV1 mAb as defined by the loss of reactivity to the peptides lacking the residues. P39: SEQ ID NO: 26; P40: SEQ ID NO: 27; P41: SEQ ID NO: 28; P42: SEQ ID NO: 29; P43: SEQ ID NO: 30; P44: SEQ ID NO: 31; P45: SEQ ID NO: 32; P46: SEQ ID NO: 33; P47: SEQ ID NO: 34; P31a: SEQ ID NO: 35; P24a: SEQ ID NO: 36; P25a: SEQ ID NO: 37; P7: SEQ ID NO: 25; P51: SEQ ID NO: 38; P50: SEQ ID NO: 39; P49: SEQ ID NO: 40; P48: SEQ ID NO: 41; P38: SEQ ID NO: 42; P37: SEQ ID NO: 43; P36: SEQ ID NO: 44; P35: SEQ ID NO: 45; P34: SEQ ID NO: 46; P33: SEQ ID NO: 47; P32: SEQ ID NO: 48; P31: SEQ ID NO: 49; P30: SEQ ID NO: 50; and P29: SEQ ID NO: 51. (D) Barplots showing the average absorbance from duplicate assays quantitating the binding of 1 μg of each peptide per well to 20 ng/mL of ETV1 mAb. (E) Peptide sequences showing consecutive alanine substitution to each of the amino acid of the immunogenic peptide P1 (SEQ ID NO: 19), from the first residue in P2 to the last residue in P28. P2: SEQ ID NO: 52; P3: SEQ ID NO: 53; P4: SEQ ID NO: 54; P5: SEQ ID NO: 55; P6: SEQ ID NO: 56; P7: SEQ ID NO: 57; P8: SEQ ID NO: 58; P9: SEQ ID NO: 59; P10: SEQ ID NO: 60; P11: SEQ ID NO: 61; P12: SEQ ID NO: 62; P13: SEQ ID NO: 63; P14: SEQ ID NO: 64; P15: SEQ ID NO: 65; P16: SEQ ID NO: 66; P17: SEQ ID NO: 67; P18: SEQ ID NO: 68; P19: SEQ ID NO: 69; P20: SEQ ID NO: 70; P21: SEQ ID NO: 71; P22: SEQ ID NO: 72; P23: SEQ ID NO: 73; P24: SEQ ID NO: 74; P25: SEQ ID NO: 75; P26: SEQ ID NO: 76; P27: SEQ ID NO: 77; P28: SEQ ID NO: 78; and the sequence under P28: SEQ ID NO: 19. (F) Barplots showing the average absorbance from duplicate assays quantitating the binding of 500 ng per well of each alanine substituted peptide to 40 ng/mL ETV1 mAb. HRP Goat anti-rabbit Ig was applied at 1:16,000 dilution in all reactions.

FIG. 4 shows Surface Plasmon Resonance imaging (SPRi) binding kinetics of the 29E4 mAb to the immunogenic peptides. (A) Monitoring of the activation of the CM5 chip and immobilization of the ETV1 (29E4) antibody as ligand through Fc4 sample channel over time. (B) SPRi Fitting curves for binding of the ETV1 peptide to the ETV1 mAb. Different concentrations of the peptide, from 250 nM to 1.953 nM, were injected over the sensor surface bound with the mAb. A 1:1 binding model was used to measure the steady state binding affinity and kinetics.

FIG. 5 shows IHC staining of tissue cores of representative TMA cases with ETV1 positive expression. Tissue cores from case 001-G3C and case 002-I9E stained with H&E (left), ERG (middle) and ETV1 (right). In the first case, glands with ERG negative and ETV1 positive staining on the top panel (A to C) are enlarged (D to F) to highlight cells without ERG expression (E) but expressing ETV1 (F, black arrowhead). In the second case, glands with positive expression for ERG (top rectangle) or ETV1 (bottom rectangle) in panels G to I are enlarged to highlight ERG positive (green arrowhead) but ETV1 negative glands in panels J to L, and ERG negative but ETV1 positive glands in panels M to O (black arrowhead).

FIG. 6 shows whole mounted sections of a case with both ERG and ETV1 expression. Consecutive whole-mounted sections were stained with H&E (A) and monoclonal antibodies against ERG (B) and ETV1 (C). Glands expressing both ERG and ETV1 (boxes 1 and 2) or ETV alone (box 3) shown in the whole mounted sections in the top panels were enlarged at 4× (left panels) and 10× magnification (right panels). ERG positive cells in glands located at the transition area of collision tumors were indicated by green arrowheads (E, K, H and N). Endothelial cells stained by ERG mAb are indicated by blue arrowheads (K and N). ETV1 positive cells in glands located at the transition area of collision tumors (F, L, I and O) as well as exclusively ETV1 positive glands (R and U) were indicated by black arrowheads.

### DETAILED DESCRIPTION

Reference will now be made in detail to various exemplary embodiments, examples of which are illustrated in the accompanying drawings and discussed in the detailed description that follows. It is to be understood that the following detailed description is provided to give the reader a fuller understanding of certain embodiments, features, and details of aspects of the disclosure, and should not be interpreted as limiting the scope of the disclosure.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods and/or to make and use the disclosed compositions. When “about” is present before a series of numbers or a range, it is understood that “about” can modify each of the numbers in the series or range.

The term “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “antibody” or “antibodies” as used in this disclosure refers to an immunoglobulin or an antigen-binding fragment thereof. As will be understood by those in the art, the immunological binding reagents encompassed by the term “antibody” or “antibodies” extend to all antibodies from all species, and antigen binding fragments thereof and include, unless otherwise specified, polyclonal, monoclonal, monospecific, bispecific, polyspecific, humanized, human, camelised, mouse, non-human primates, single-chain, chimeric, synthetic, recombinant, hybrid, mutated, CDR-grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda.

The term “antigen” refers to any substance that is capable of generating an immune response (e.g., the production of antibodies).

The term “antigen-binding fragment” refers to a part of an antibody molecule that comprises amino acids responsible for the specific binding between antibody and antigen. For certain antigens, the antigen-binding fragment may only bind to a part of the antigen. The part of the antigen that is specifically recognized and bound by the antibody is referred to as the “epitope” or “antigenic determinant.” Antigen-binding fragments include Fab (Fragment antigen binding); a F(ab′)2 fragment, a bivalent fragment having two Fab fragments linked by a disulfide bridge at the hinge region; Fv fragment; a single chain Fv fragment (scFv) (see e.g., Bird et al. (1988) Science 242:423-426; and Huston et al. (1988) Proc. Natl. Acad. Sci. USA 85:5879-5883); a Fd fragment having the two VH and CH1 domains; dAb (Ward et al., (1989) Nature 341:544-546), and other antibody fragments that retain antigen binding function. The Fab fragment has VH CH1 and VL CL domains covalently linked by a disulfide bond between the constant regions. The Fv fragment is smaller and has VH and VL domains non-covalently linked. To overcome the tendency of noncovalently linked domains to dissociate, a scFv can be constructed. The scFv contains a flexible polypeptide that links (1) the C terminus of VH to the N terminus of VL, or (2) the C terminus of VL to the N terminus of VH. A 15-mer (Gly4Ser)3 peptide may be used as a linker, but other linkers are known in the art. These antibody fragments are obtained using conventional techniques known to those with skill in the art, and the fragments are evaluated for function in the same manner as are intact antibodies.

The term “at least” prior to a number or series of numbers (e.g., “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

As used herein, the terms “binds” or “binding” refer to the interaction between an antibody, or an antigen-binding fragment, and an antigen, or an antigenic fragment.

The term “diagnosing” or “diagnosis” as used herein refers to the use of information (e.g., antibody binding or data from tests on biological samples, signs and symptoms, physical exam findings, cognitive performance results, etc.) to anticipate the most likely outcomes, timeframes, and/or response to a particular treatment for a given disease, disorder, or condition, based on comparisons with a plurality of individuals sharing common nucleotide sequences, symptoms, signs, family histories, or other data relevant to consideration of a patient's health status.

The term “effective amount” refers to a dosage or amount that is sufficient for treating an indicated disease or condition.

The term “identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). Typical methods to determine identity are designed to give the largest match between the sequences tested. Methods to determine identity and similarity are codified in publicly available computer programs. Typical computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

The term “immunoassay” refers to any assay that uses at least one specific antibody for the detection and/or quantification of an antigen. Immunoassays include, but not limited to, rapid strip tests, Western blots, enzyme-linked immunosorbent assays (ELISAs), radio-immunoassays, and immunofluorescence assays and any other antigen-antibody reactions including, for example, “flocculation” (i.e., a colloidal suspension produced upon the formation of antigen-antibody complexes), “agglutination” (i.e., clumping of cells or other substances upon exposure to antibody), “particle agglutination” (i.e., clumping of particles coated with antigen in the presence of antibody or the clumping of particles coated with antibody in the presence of antigen), “complement fixation” (i.e., the use of complement in an antibody-antigen reaction method), and other methods commonly used in serology, immunology, immunocytochemistry, immunohistochemistry, and related fields.

The term “in need thereof” means that the subject has been identified or suspected as having a need for the particular method or treatment. In some embodiments, the identification can be by any means of diagnosis or observation. In any of the methods described herein, the subject can be in need thereof. In some embodiments, the subject in need thereof is a human suspected of having a cancer associated with overexpression of ETV1. In some embodiments, the subject in need thereof is a human diagnosed with cancer. In some embodiments, the subject in need thereof is a human seeking treatment for cancer. In some embodiments, the subject in need thereof is a human undergoing treatment for cancer.

As used herein, the term “in some embodiments,” “in other embodiments,” or the like, refers to embodiments of all aspects of the disclosure, unless the context clearly indicates otherwise.

The term “kit” refers to a combination of reagents and/or apparatus, which facilitates sample analysis. In some embodiments, a kit may further include one or more apparatus to facilitate sample harvesting. In some embodiments, a kit may further include one or more reagents for sample processing. In some embodiments, a kit may further include one or more written instructions.

The term “sample” is used herein in the broadest sense and can be obtained from any source in the body. A sample can encompass fluids, solids and/or tissues. In some embodiments, a sample obtained from a subject may include, but is not limited to, any or all of the following: a cell or cells, a portion of tissue, blood, serum, ascites, urine, saliva, and other body fluids, secretions, or excretions. The term “sample” also includes any material derived by processing such a sample, such as nucleotide molecules or polypeptides extracted from the sample.

The terms “subject,” “host,” “patient,” and “individual” are used interchangeably herein to refer to any subject for whom diagnosis or therapy is desired, particularly mammals, such as humans.

The terms “treatment” or “treating” and the like refer to any treatment of any disease or condition in an animal, such as a bird or mammal, e.g. particularly a human or a mouse, and includes inhibiting a disease, condition, or symptom of a disease or condition, e.g., arresting its development and/or delaying its onset or manifestation in the patient or relieving a disease, condition, or symptom of a disease or condition, e.g., causing regression of the condition or disease and/or its symptoms.

### ETV1 (ETS Variant Transcription Factor 1)

ETV1 is a transcription factor belonging to the large ETS (E-twenty six) family and PEA3 subfamily. ETS family members are defined by a highly conserved DNA binding domain, the ETS domain, which is a winged helix-turn-helix structure that binds to DNA sites with a central GGA DNA sequence. Multiple ETS factors have been found to be associated with cancer. In a condition called Ewing's sarcoma, the ERG ETS transcription factor is fused to the EWS gene (Ida et al, 1995, Int. J. Cancer, 63(4):500-504). The fusion of TEL to the JAK2 protein results in early pre-B acute lymphoid leukemia (Peeters et al, 1997, Blood, 90(7):2535-2540) and ERG and ETV1 are ETS family members which are overexpressed, and whose gene fusions are found, in prostate cancer (Tomlins et al, 2005, Science, 310(5748):644-648).

In gastrointestinal stromal tumors (GISTs), ETV1 is universally highly expressed. Activated KIT, through MEK, prolongs ETV1 protein stability and cooperates with ETV1 to promote tumorigenesis GIST arises from interstitial cells of Cajal (ICCs) exhibiting high levels of endogenous ETV1 expression that, when coupled with an activating KIT mutation, drives an oncogenic ETS transcriptional program. This oncogenic role for ETV1 in GIST differs from classical models of other ETS-dependent tumors such as prostate cancer, melanoma and Ewing's sarcoma where genomic translocation or amplification drives aberrant ETS expression and promote tumorigenesis (Tomlins et al, 2005, Science, 310(5748):644-648; Mertens et al, 2009, Semin. Oncol., 26:312-323; Jane-Valbuena et al, 2010, Cancer Res., 70:2075-2084).

Human ETV1 is a protein of 477 amino acids in length and comprises the amino acid sequence of SEQ ID NO: 17.

Human ETV1 (Genbank Accession No. P50549; Uniprot Accession No. P50549-1; SEQ ID NO: 17)

MDGFYDQQVPYMVTNSQRGRNCNEKPTNVRKRKFINRDLAHDSEELFQDLSQLQETW
LAEAQVPDNDEQFVPDYQAESLAFHGLPLKIKKEPHSPCSEISSACSQEQPFKFSYGEKCL
YNVSAYDQKPQVGMRPSNPPTPSSTPVSPLHHASPNSTHTPKPDRAFPAHLPPSQSIPDSS
YPMDHRFRRQLSEPCNSFPPLPTMPREGRPMYQRQMSEPNIPFPPQGFKQEYHDPVYEH
NTMVGSAASQSFPPPLMIKQEPRDFAYDSEVPSCHSIYMRQEGFLAHPSRTEGCMFEKGP
RQFYDDTCVVPEKFDGDIKQEPGMYREGPTYQRRGSLQLWQFLVALLDDPSNSHFIAW
TGRGMEFKLIEPEEVARRWGIQKNRPAMNYDKLSRSLRYYYEKGIMQKVAGEYVYKF
VCDPEALFSMAFPDNQRPLLKTDMERHINEEDTVPLSHFDESMAYMPEGGCCNPHPYN
EGYVY

### Monoclonal ETV1 Antibodies

There currently exists a need for effective monoclonal ETV1 antibodies for use in diagnostic and therapeutic applications. Polyclonal ETV1 antibodies (e.g., rabbit polyclonal antibody commercially available from Abcam and Covance) show reasonable ETV1 specificity on Western Blots (with multiple nonspecific bands) and limited utility (dirty) for immunohistochemistry (IHC). In addition to performing poorly in IHC tests, polyclonal antibodies have limited usefulness in human therapeutics and diagnostics. The usefulness of monoclonal antibodies stems from their specificity of binding, their homogeneity, and their ability to be produced in unlimited quantities from cultured hybridoma. Provided herein are monoclonal antibodies and antigen-binding fragments thereof that specifically bind to an epitope of ETV1.

In some embodiments, the monoclonal antibodies or antigen-binding fragments thereof of the disclosure comprise a heavy chain variable domain comprising a complementarity determining region 1 (CDR1) comprising the amino acid sequence of SEQ ID NO: 3, a CDR2 comprising the amino acid sequence of SEQ ID NO: 4, and a CDR3 comprising the amino acid sequence of SEQ ID NO: 5. In some embodiments, the monoclonal antibodies or antigen-binding fragments thereof of the disclosure comprise a light chain variable domain comprising a CDR1 comprising the amino acid sequence of SEQ ID NO: 6, a CDR2 comprising the amino acid sequence of SEQ ID NO: 7, and a CDR3 comprising the amino acid sequence of SEQ ID NO: 8. In some embodiments, the monoclonal antibodies or antigen-binding fragments thereof of the disclosure comprise a heavy chain variable domain comprising a CDR 1 comprising the amino acid sequence of SEQ ID NO: 3, a CDR2 comprising the amino acid sequence of SEQ ID NO: 4, and a CDR3 comprising the amino acid sequence of SEQ ID NO: 5, and a light chain variable domain comprising a CDR1 comprising the amino acid sequence of SEQ ID NO: 6, a CDR2 comprising the amino acid sequence of SEQ ID NO: 7, and a CDR3 comprising the amino acid sequence of SEQ ID NO: 8.

In some embodiments, the monoclonal antibodies or antigen-binding fragments thereof of the disclosure comprise a heavy chain variable domain comprising a CDR1 comprising the amino acid sequence of SEQ ID NO: 9, a CDR2 comprising the amino acid sequence of SEQ ID NO: 10, and a CDR3 comprising the amino acid sequence of SEQ ID NO: 11. In some embodiments, the monoclonal antibodies or antigen-binding fragments thereof of the disclosure comprise a light chain variable domain comprising a CDR1 comprising the amino acid sequence of SEQ ID NO: 12, a CDR2 comprising the amino acid sequence of SEQ ID NO: 13, and a CDR3 comprising the amino acid sequence of SEQ ID NO: 14. In some embodiments, the monoclonal antibodies or antigen-binding fragments thereof of the disclosure comprise a heavy chain variable domain comprising a CDR1 comprising the amino acid sequence of SEQ ID NO: 9, a CDR2 comprising the amino acid sequence of SEQ ID NO: 10, and a CDR3 comprising the amino acid sequence of SEQ ID NO: 11, and a light chain variable domain comprising a CDR1 comprising the amino acid sequence of SEQ ID NO: 12, a CDR2 comprising the amino acid sequence of SEQ ID NO: 13, and a CDR3 comprising the amino acid sequence of SEQ ID NO: 14. CDRs of the heavy and light chains of an antibody can be identified using algorithms known in the art, including but not limited to, the Kabat-Chothia system (bioinf.org.uk/abs/info.html) and the Ofran method (ofranlab.org/paratome/; Kunik et al., PLoS Comput., 2012, 8(2): e1002388; Kunik et al., Nucleic Acids Res., 2012, 40:W521-4).

In some embodiments, the monoclonal antibodies or antigen-binding fragments thereof of the disclosure comprise a heavy chain variable domain comprising the amino acid sequence SEQ ID NO: 1. In some embodiments, the monoclonal antibodies or antigen-binding fragments thereof of the disclosure comprise a light chain variable domain comprising the amino acid sequence SEQ ID NO: 2. In some embodiments, the monoclonal antibodies or antigen-binding fragments thereof of the disclosure comprise a heavy chain variable domain comprising the amino acid sequence SEQ ID NO: 1 and a light chain variable domain comprising the amino acid sequence SEQ ID NO: 2.

Modified versions of the monoclonal antibodies or antigen-binding fragments thereof of the disclosure are also provided. Typically, modifications to an antibody can be introduced through the nucleic acids that encode variable domains of the antibody. These modifications can include deletions, insertions, point mutations, truncations, and amino acid substitutions and addition of amino acids or non-amino acid moieties. For example, random mutagenesis of the disclosed variable domain sequences can be used to generate variant variable domains still capable of binding ETV1. A technique using error-prone PCR is described by Gram et al. (Proc. Nat. Acad. Sci. U.S.A., 1992, 89:3576-3580). Another method uses direct mutagenesis of the disclosed variable domain sequences. Modifications can also be made directly to the amino acid sequence, such as by cleavage, addition of a linker molecule or addition of a detectable moiety, such as biotin, addition of a fatty acid, and the like.

Accordingly, in some embodiments, the monoclonal antibodies or antigen-binding fragments thereof of the disclosure comprise a heavy chain variable domain comprising an amino acid sequence that is at least about 90%, 95%, 96%, 97%, 98%, or 99% identical to the amino acid sequence of SEQ ID NO: 1. In some embodiments, the monoclonal antibodies or antigen-binding fragments thereof of the disclosure comprise a light chain variable domain comprising an amino acid sequence that is at least about 90%, 95%, 96%, 97%, 98%, or 99% identical to the amino acid sequence of SEQ ID NO: 2. In some embodiments, the monoclonal antibodies or antigen-binding fragments thereof of the disclosure comprise a heavy chain variable domain comprising an amino acid sequence that is at least about 90%, 95%, 96%, 97%, 98%, or 99% identical to the amino acid sequence of SEQ ID NO: 1 and a light chain variable domain comprising an amino acid sequence that is at least about 90%, 95%, 96%, 97%, 98%, or 99% identical to the amino acid sequence of SEQ ID NO: 2.

In some embodiments, the monoclonal antibodies or antigen-binding fragments thereof of the disclosure comprise a heavy chain variable domain that is identical to the amino acid sequence of SEQ ID NO: 1 except for 1, up to 2, up to 3, up to 4, up to 5, up to 6, up to 7, and in certain cases, up to 10 amino acid modifications (e.g., substitutions). In some embodiments, the monoclonal antibodies or antigen-binding fragments thereof of the disclosure comprise a light chain variable domain that is identical to the amino acid sequence of SEQ ID NO: 2 except for 1, up to 2, up to 3, up to 4, up to 5, up to 6, up to 7, and in certain cases, up to 10 amino acid modifications (e.g., substitutions). In some embodiments, the monoclonal antibodies or antigen-binding fragments thereof of the disclosure comprise a heavy chain variable domain that is identical to the amino acid sequence of SEQ ID NO: 1 except for 1, up to 2, up to 3, up to 4, up to 5, up to 6, up to 7, and in certain cases, up to 10 amino acid modifications (e.g., substitutions) and a light chain variable domain that is identical to the amino acid sequence of SEQ ID NO: 2 except for 1, up to 2, up to 3, up to 4, up to 5, up to 6, up to 7, and in certain cases, up to 10 amino acid modifications (e.g., substitutions). In some embodiments, the modified amino acids are located within at least the CDR regions. In some embodiments, the modifications are located outside the CDR regions. Typically, modification of the amino acid sequence involves substitution of an amino acid with an amino acid having similar charge, hydrophobic, or stereochemical characteristics. More drastic substitutions in regions outside of the CDRs may also be made as long as they do not adversely affect (e.g., reduce affinity by more than 50% as compared to unsubstituted antibody) the binding properties of the antibody.

Modified versions of the monoclonal antibodies or antigen-binding fragments thereof of the disclosure can also be screened to identify which mutation provides a modified antibody or antigen-binding fragment thereof that retains a desired property, such as a higher binding affinity than the parent antibody for ETV1.

The monoclonal antibodies or antigen-binding fragments thereof of the disclosure bind specifically to an epitope of ETV1 without cross-reactivity to other ETS factors, such as ETV4 and ETV5. An “epitope” is the part of the antigen that is specifically recognized and bound by the antibody. In some embodiments, the monoclonal antibodies or antigen-binding fragments thereof of the disclosure bind to an epitope of ETV1 comprising the amino acid sequence of SEQ ID NO: 15 or an immunogenic fragment thereof. In some embodiments, the monoclonal antibodies or antigen-binding fragments thereof of the disclosure bind to an epitope of ETV1 comprising the amino acid sequence of SEQ ID NO: 16 or an immunogenic fragment thereof. In certain embodiments, the epitope of ETV1 contains no more than 30 amino acids, such as no more than 29, no more than 28, no more than 27, no more than 26, no more than 25, no more than 24, no more than 23, no more than 22, no more than 21, no more than 20, no more than 19, no more than 18, no more than 17, no more than 16, no more than 15, no more than 14, no more than 13, no more than 12, no more than 11, or no more than 10 amino acids.

Another aspect is related to compositions, such as immunogenic compositions, comprising an epitope of ETV1, such as the epitope of ETV1 comprising the amino acid sequence of SEQ ID NO: 15 or SEQ ID NO: 16. In some embodiments, the epitope comprises the amino acid of SEQ ID NO: 16 and contains no more than 30 amino acids. In some embodiments, the epitope comprises the amino acid sequence of SEQ ID NO: 15 and contains no more than 15 amino acids. In some embodiments, the composition further comprises an adjuvant and/or a hapten, such as KLH or ovalbumin. These compositions can be used, for example, in a method of producing antibodies. Accordingly, in some embodiments, provided herein are methods of generating a monoclonal antibody that binds to ETV1, the method comprising administering the immunogenic composition disclosed herein to a non-human mammal, isolating B cells from the non-human mammal, immortalizing the B cells to create a cell line capable of producing a monoclonal antibody, and selecting the monoclonal antibody that binds to the epitope of ETV1 comprising the amino acid sequence of SEQ ID NO: 15 or SEQ ID NO: 16. For example, the monoclonal antibodies or antigen-binding fragments thereof of the disclosure can be produced by first immunizing rabbits with immunogenic peptides of ETV1, such as a peptide comprising the amino acid sequence of SEQ ID NO: 15 or SEQ ID NO: 16, followed by selecting one or more hybridoma clones that produce the most specific IgG subtype monoclonal antibody against ETV1 for further expansion as described in Example 1 below. In some embodiments therefore, the monoclonal antibodies or antigen-binding fragments thereof of the disclosure are rabbit monoclonal antibodies or antigen-binding fragments thereof.

Also provided herein are nucleic acid molecules encoding the monoclonal antibodies or antigen-binding fragments thereof of the disclosure, as well as host cells comprising such nucleic acid molecules.

In some embodiments, when the monoclonal antibodies or antigen-binding fragments thereof of the disclosure are used for therapeutic purposes, it may prove advantageous to use a humanized or veneered antibody to reduce any potential immunogenic reaction. In general, humanized or veneered antibodies minimize unwanted immunological responses that limit the duration and effectiveness of therapeutic applications of non-human antibodies in human recipients.

A number of methods for preparing humanized antibodies comprising an antigen binding portion derived from a non-human antibody have been described in the art. In particular, antibodies with rodent variable regions and their associated complementarity-determining regions (CDRs) fused to human constant domains have been described (see e.g., Winter et al., Nature, 1991, 349:293; Lobuglio et al., Proc. Nat. Acad. Sci. USA, 1989, 86:4220; Shaw et al., J. Immunol., 1987, 138:4534; and Brown et al., Cancer Res., 1987, 47:3577). Rodent CDRs grafted into a human supporting framework region (FR) prior to fusion with an appropriate human antibody constant domain (see e.g., Riechmann et al., Nature, 1988, 332:323; Verhoeyen et al., Science, 1988, 239:1534; and Jones et al., Nature, 1986, 321:522) and rodent CDRs supported by recombinantly veneered rodent FRs have also been described (see e.g., EP0519596).

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes (see e.g., Lonberg and Huszar, Int. Rev. Immunol., 1995, 13:65-93 and U.S. Pat. Nos. 5,545,806; 5,569,825; 5,625,126; 5,633,425; and 5,661,016).

Veneered versions of the monoclonal antibodies or antigen-binding fragments thereof of the disclosure may also be used in the methods disclosed herein. The process of veneering involves selectively replacing FR residues from, e.g., a murine heavy or light chain variable region, with human FR residues in order to provide an antibody that comprises an antigen binding portion which retains substantially all of the native FR protein folding structure. Veneering techniques are based on the understanding that the antigen binding characteristics of an antigen binding portion are determined primarily by the structure and relative disposition of the heavy and light chain CDR sets within the antigen-association surface (see e.g., Davies et al., Ann. Rev. Biochem., 1990, 59:439). Thus, antigen association specificity can be preserved in a humanized antibody only wherein the CDR structures, their interaction with each other and their interaction with the rest of the variable region domains are carefully maintained. By using veneering techniques, exterior (e.g., solvent-accessible) FR residues which are readily encountered by the immune system are selectively replaced with human residues to provide a hybrid molecule that comprises either a weakly immunogenic, or substantially non-immunogenic veneered surface.

### Pharmaceutical Compositions

Also provided are compositions, such as pharmaceutical compositions, comprising one or more monoclonal antibodies or antigen-binding fragments thereof of the disclosure. In some embodiments, the disclosure provides at least one monoclonal antibody, or an antigen-binding fragment thereof, described herein and at least one pharmaceutically acceptable excipient. Such pharmaceutical compositions may optionally comprise and/or be administered in combination with one or more additional therapeutically active substances. In some embodiments, the disclosed pharmaceutical compositions are useful in medicine. In some embodiments, the disclosed pharmaceutical compositions are formulated for administration to humans.

The pharmaceutical compositions provided herein may be provided in a sterile injectable form (e.g., a form that is suitable for subcutaneous injection or intravenous infusion). For example, in some embodiments, the disclosed pharmaceutical compositions are provided in a liquid dosage form that is suitable for injection. In some embodiments, the disclosed pharmaceutical compositions are provided as powders (e.g., lyophilized and/or sterilized), optionally under vacuum, which are reconstituted with an aqueous diluent (e.g., water, buffer, salt solution, etc.) prior to injection. In some embodiments, the disclosed pharmaceutical compositions are diluted and/or reconstituted in water, sodium chloride solution, sodium acetate solution, benzyl alcohol solution, phosphate buffered saline, etc.

In some embodiments, the disclosed pharmaceutical compositions comprise one or more pharmaceutically acceptable excipients. As used herein, pharmaceutically acceptable excipients may be or comprise solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro, (Lippincott, Williams & Wilkins, Baltimore, MD, 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of the present disclosure.

The disclosed pharmaceutical compositions may be prepared by any method known or hereafter developed in the art of pharmacology. In some embodiments, such preparatory methods include the step of bringing active ingredient into association with one or more excipients and/or one or more other accessory ingredients, and then, if necessary and/or desirable, shaping and/or packaging the product into a desired single- or multi-dose unit.

### Uses of Monoclonal Antibodies

The monoclonal antibodies or antigen-binding fragments thereof of the disclosure can be used for diagnostic and therapeutic applications. Their ability to specifically bind ETV1 without cross-reactivity to other ETS factors, such as ETV4 and ETV5, makes it possible to use ETV1 as a diagnostic marker and therapeutic target. Accordingly, in some embodiments, provided herein is a method for determining the likelihood that a subject has a cancer, the method comprising a) determining a level of ETV1 expression in a sample obtained from the subject using the monoclonal antibody or the antigen-binding fragment thereof of the disclosure, and b) comparing the level of expression of ETV1 obtained in step a) with a reference level of ETV1 expression, wherein the reference level of ETV1 expression is obtained from a subject known not to have a cancer and an increased level of ETV1 expression in the sample as compared to the reference level of ETV1 expression indicates that there is an increased likelihood that the subject has a cancer. Alternatively, if the reference level of ETV1 expression is obtained from a subject known to have a cancer, then a decreased level of ETV1 expression in the sample as compared to the reference level of ETV1 expression may indicate that there is an increased likelihood that the subject does not have a cancer. In some embodiments, the level of ETV1 expression in the sample is determined using an analysis selected from the group consisting of Western blotting, protein gel electrophoresis, immunoprecipitation, ELISA, immunohistochemistry, and combinations thereof. In some embodiments, the level of ETV1 expression in the sample is determined by performing immunohistochemistry on the sample. In some embodiments, the subject is a human suspected of having a cancer. In some embodiments, the cancer is prostate cancer, gastrointestinal stromal tumor, small round cell sarcoma, melanoma, or gliomas.

Also provided herein is a method of diagnosing a cancer associated with overexpression of ETV1 in a subject in need thereof, the method comprising a) determining a level of ETV1 expression in a sample obtained from the subject using the monoclonal antibody or the antigen-binding fragment thereof of the disclosure, and b) comparing the level of ETV1 expression obtained in step a) with a reference level of ETV1 expression, wherein the reference level of ETV1 expression is obtained from a subject known not to have a cancer and an increased level of ETV1 expression in the sample as compared to the reference level of ETV1 expression indicates that there is an increased likelihood that the subject has a cancer, and/or wherein the reference level of ETV1 expression is obtained from a subject known to have a cancer associated with overexpression of ETV1 and a decreased level of ETV1 expression in the sample as compared to the reference level of ETV1 expression indicates that there is an increased likelihood that the subject does not have a cancer. In some embodiments, the level of ETV1 expression in the sample is determined using an analysis selected from the group consisting of Western blotting, protein gel electrophoresis, immunoprecipitation, ELISA, immunohistochemistry, and combinations thereof. In some embodiments, the level of ETV1 expression in the sample is determined by performing immunohistochemistry on the sample. In some embodiments, the subject is a human suspected of having a cancer associated with overexpression of ETV1. In some embodiments, the cancer associated with overexpression of ETV1 is prostate cancer, gastrointestinal stromal tumor, small round cell sarcoma, melanoma, or gliomas.

The monoclonal antibodies or antigen-binding fragments thereof of the disclosure can also be used for therapeutic application. Accordingly, in some embodiments, disclosed herein is a method of treating a cancer associated with overexpression of ETV1, the method comprising administering to a subject in need thereof a therapeutically effective amount of the monoclonal antibodies or antigen-binding fragments thereof of the disclosure, or a pharmaceutical composition comprising such monoclonal antibodies or antigen-binding fragments thereof. In such applications, the monoclonal antibodies or antigen-binding fragments thereof of the disclosure, or a pharmaceutical composition comprising the same, can be administered to the subject as a monotherapy or as part of a combination therapy. In some embodiments, the cancer associated with overexpression of ETV1 is prostate cancer, gastrointestinal stromal tumor, small round cell sarcoma, melanoma, or gliomas.

The monoclonal antibodies or antigen-binding fragments thereof of the disclosure can also be used for detection applications. Accordingly, also provided herein is a method of detecting ETV1 expression in a sample, the method comprising incubating the sample with the monoclonal antibody or the antigen-binding fragment thereof disclosed herein under conditions permitting the detection of ETV1 expression when ETV1 is present in the sample. In some embodiments, the ETV1 expression is detected using an analysis selected from the group consisting of Western blotting, protein gel electrophoresis, immunoprecipitation, ELISA, immunohistochemistry, and combinations thereof. In some embodiments, the sample is a biological sample comprising cells. In such applications, the monoclonal antibodies or antigen-binding fragments thereof of the disclosure can be conjugated to at least one detection entity to allow detection of ETV1 protein in a sample. For example, the monoclonal antibodies or antigen-binding fragments thereof of the disclosure can be conjugated with tracer chemicals for use in clinical imaging. A variety of detectable agents can be used as detection entity (e.g., labeling moieties) in conjunction with the monoclonal antibodies or antigen-binding fragments thereof of the disclosure.

A detection entity may be directly detectable or indirectly detectable. Examples of detection entity include, but are not limited to, various ligands, radionuclides (e.g., ³H, ¹⁴C, ¹⁸F, ¹⁹F, ³²P, ³⁵S, ¹³⁵I, ¹²³I, ⁶⁴Cu, ¹⁸⁷Re, ¹¹¹In, ⁹⁰Y, ⁹⁹mTc, ¹⁷⁷Lu, etc.), fluorescent dyes (for specific exemplary fluorescent dyes, see below), chemiluminescent agents (such as, for example, acridinium esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, digoxigenin, haptens, and proteins for which antisera or monoclonal antibodies are available.

In some embodiments, a detection entity comprises a fluorescent label. Numerous known fluorescent labeling moieties of a variety of chemical structures and physical characteristics are suitable for use as the detection entity in the present disclosure. Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, β carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, Spectrum Red™, Spectrum Green™, cyanine dyes (e.g., Cy-3™, Cy-5™, Cy-3.5™, Cy-5.5™ etc.), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660, Alexa Fluor 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. For more examples of suitable fluorescent dyes and methods for coupling fluorescent dyes to other chemical entities such as proteins and peptides, see, for example, “Handbook of Fluorescent Probes and Research Products” by R. P. Haugland, Molecular Probes, Inc., Eugene, OR.

In some embodiments, a detection entity comprises an enzyme. Examples of suitable enzymes include, but are not limited to, those used in an ELISA, e.g., horseradish peroxidase, beta-galactosidase, luciferase, alkaline phosphatase, etc. Other examples include beta-glucuronidase, beta-D-glucosidase, urease, glucose oxidase, etc. An enzyme may be conjugated to a targeting entity (e.g., chlorotoxin moiety) using a linker group such as a carbodiimide, a diisocyanate, a glutaraldehyde, and the like. More detailed description of suitable linkers is provided elsewhere herein.

In some embodiments, a detection entity comprises a radioisotope that is detectable by Single Photon Emission Computed Tomography (SPECT) or Position Emission Tomography (PET). Examples of such radionuclides include, but are not limited to, iodine-131 (¹³¹I), iodine-125 (¹²¹I), bismuth-212 (²¹²Bi), bismuth-213 (²¹³Bi), astatine-221 (²¹¹At), copper-67 (⁶⁷Cu), copper-64 (⁶⁴Cu), rhenium-186 (¹⁸⁶Re), rhenium-188 (¹⁸⁸Re), phosphorus-32 (³²P), samarium-153 (¹⁵³Sm), lutetium-177 (¹¹⁷Lu), technetium-99m (⁹⁹mTc), gallium-67 (⁶⁷Ga), indium-I111 (¹¹¹In), and thallium-201 (²⁰¹Tl).

In some embodiments, a detection entity comprises a paramagnetic metal ion that is a good contrast enhancer in Magnetic Resonance Imaging (MRI). Examples of such paramagnetic metal ions include, but are not limited to, gadolinium III (Gd³⁺), chromium III (Cr³⁺), dysprosium III (Dy³⁺), iron III (Fe³⁺), manganese II (Mn²⁺), and ytterbium HI (Yb³⁺).

### Kits

The disclosure also provides a variety of kits for conveniently and/or effectively carrying out any of the methods disclosed herein. Kits of the disclosure typically comprise one or more of the monoclonal antibodies or antigen-binding fragments thereof described herein for detecting ETV1 expression in a sample. In some embodiments, kits of the disclosure comprise the monoclonal antibodies or antigen-binding fragments thereof described herein in a container. Other kit embodiments include one or more reagents for detection of the monoclonal antibodies or antigen-binding fragments thereof, such as secondary antibodies. In some such instances, the secondary antibody is directly labeled with a detectable moiety (as described elsewhere in this disclosure). In other instances, the primary or secondary (or higher-order) antibody is conjugated to a hapten (such as biotin, DNP, DIG, etc.), which is detectable by a detectably labeled cognate hapten-binding molecule (e.g., streptavidin (SA)-horse radish peroxidase, SA-alkaline phosphatase, SA-QDot® (Invitrogen, Carlsbad, CA), etc.). In some embodiments, the primary or secondary antibody is conjugated with a fluorescent detection moiety (e.g., FITC, rhodamine, ALEXA FLUOR® (Invitrogen, Carlsbad, CA) dyes, Cy designated fluorophores, etc.). Some kit embodiments may include colorimetric reagents (e.g., DAB, AEC, etc.) in suitable containers to be used in concert with primary or secondary (or higher-order) antibodies that are labeled with enzymes for the development of such colorimetric reagents.

In some embodiments, kits of the disclosure further comprise a control. In some embodiments, kits of the disclosure also comprise instructions for carrying out an immunoassay for determination of ETV1 expression, such as instructions for processing samples, for performing tests, for interpreting results, for solubilizing ETV1 antibodies, and/or for storage of ETV1 antibodies. In some embodiments, kits of the disclosure can also comprise buffers and/or other reagents necessary for performing tests. In some embodiments, kits of the disclosure can comprise panels of antibodies. Other components of kits may include cells, cell culture media, tissue, and/or tissue culture media.

### EXAMPLES

The examples provided below are simply for illustrative purposes. Those of skill in the art will be able to readily determine appropriate methods and equipment in order to produce suitable lipid particles as described herein.

### Example 1. Development and Characterization of an ETV1 Rabbit Monoclonal Antibody for the Immunohistochemical Detection of ETV1 Expression in Cancer Tissue Specimens

Gene rearrangements of ETV1 in prostate cancer (PCa), round cell sarcomas, gastrointestinal stromal tumors and other cancers frequently result in ETV1 protein overexpression. ETV1 gene fusions, occurring in approximately 5% of PCa, are associated with poor outcome and are presumably mutually exclusive from TMPRSS2-ERG fusion or PTEN deletion. The lack of ETV1 specific monoclonal antibodies (mAb) has limited the ability to detect ETV1-positive tumors and understand its oncogenic function in various cancers.

Immunization of rabbits using an antigenic ETV1 peptide followed by ELISA and immunoblot screening identified a hybridoma (clone 29E4) that produced a highly selective mAb without cross-reactivity to other ETS factors. Key residues required for mAb binding were probed by ELISA. The binding kinetics of the antibody to its immunogenic peptide was measured by surface plasmon resonance imaging (SPRi). Its ability to detect ETV1 expression by immunohistochemistry (IHC) assay was assessed together with the ERG 9FY mAb using a PCa tissue microarray (TMA) and whole-mounted prostate sections.

We identified a minimal epitope required for efficient mAb binding between amino acids 215 to 228 (MSEPNIPFPPQGFK; SEQ ID NO: 16), with two phenylalanine residues as core epitopes. SPRi measurement of the mAb revealed a high affinity constant. ETV1(+) tumors were detected in five of the 100 TMA cases, including one case with hybrid ERG(+) and ETV1(+) glands. Examination of ETV1 expression in a whole-mounted prostate section revealed glands with a mosaic staining pattern of cells that are partly positive and interspersed with ETV1(−) cells.

The high binding affinity and target specificity of the ETV1 29E4 mAb were demonstrated in Western blot, immunofluorescence and IHC assays. The successful detection of ETV1 expression in human prostate tissue by the antibody reveals its potential utility for the diagnosis and stratification of tumor cases with high ETV1 overexpression and in identifying patients most likely to have worse outcomes.

### 1. Methods

i. Identification of ETV1 Antigenic Regions

The potentially antigenic regions on the ETV1 protein were selected using software that predict antigenic regions including the EMBOSS antigenic software. Regions where these potentially antigenic amino acids are located were visualized using Hopp-Wood hydrophilic and Kyte-Doolittle hydrophobic plots to make sure that they are not located within the ETS DNA binding domain, which is conserved among all ETS transcription factors. The hydropathic and physiochemical properties of the peptides were further verified to ensure that they carry hydrophilic residues. The selected antigenic regions were confirmed to be non-homologous to other ETSs factors, especially between ETV5 and ETV4, which are the closest among the ETS transcription factors. The antigenic peptides were ascertained to be located at exons downstream of most gene fusion located.

ii. Antibody Production

Antibody production was performed by Abcam (Burlingame, CA) using a 27 amino acid immunogenic peptide, C-QRQMSEPNIPFPPQGFKQEYHDPVYEH (SEQ ID NO: 15), derived from amino acid residues 212 to 238 of the ETV1 protein (GenBank: AAD29877.1). A cysteine residue was added to the N-terminus position to facilitate conjugation to adjuvants. A pair of rabbits were immunized by subcutaneous injection or equal amounts of peptides conjugated with KLH, ovalbumin, and Blue Carrier protein adjuvants at two to three weeks intervals, up to a total of five injections). Pre-bleed sera and two test-bleed sera were tested by Western blot analysis with cell lysates from HEK293 cells that exogenously expressed the ETV1 protein. A rabbit displaying the most reactive sera against the ETV1 protein was selected for splenectomy, and subsequent fusion of spleen cells with plasmacytoma as described in Spieker-Polet et al. (Proc Natl Acad Sci USA, 1995, 92:9348-9352). Hybridoma clones producing antibodies against ETV1 were screened against the immunogen peptide by ELISA. Immunoreactive hybridoma supernatants were further evaluated by Western blot analysis using HEK293 cell lysates that exogenously expressed ETV1 protein. A hybridoma clone (clone 29E4) producing the most specific IgG subtype mAb against ETV1 without cross-reactivity against other ETS protein was identified for further expansion.

iii. Cell Culture and Exogenous Expression of ETS Proteins

Cell lines were purchased from the ATCC (Manassas, VA) and cultured in their respective growth medium supplemented with 10% FBS under humidified conditions at 37° C. with 5% CO₂: Human embryonic kidney (HEK) 293 cells in Eagle's Minimum Essential Medium (EMEM); LNCaP in RPMI-1640 medium; and PC3 in F-12K medium.

The pEX-NEG-M12 empty vector and expression vectors containing cDNA for ETV1 (pEX-F0572-M12-3FLAG-ETV1, NM_004956.4), ETV4 (pEX-T8074-M12-3FLAG ETV4, NM_001986.2), ETV5 (pEX-F0800-M12-3-FLAG ETV5, NM_004454.2) fused to 3FLAG epitopes at the N-terminus were purchased from Genecopoeia (Rockville, MD). ERG3 was expressed from a pCMV-ERG3 plasmid (Exons 5-16, NM_001136154) (30). The adenoviral construct expressing FLAG-FLI1 (NM_002017) fusion protein and the FLAG-SPDEF (NM_012391) expression vector were gifts from Dr. Dennis Watson.

HEK-293 cells were seeded in 15 mm tissue culture dishes at a density of 4×10⁶ cells in 20 mL of EMEM supplemented with 10% FBS to reach 50% confluency after 24 hours. Approximately 15 μg of plasmid DNA (pEX-NEG-M12 control empty vector or expression vector for ERG, ETV1, ETV4, or SPDEF) were transfected using Lipofectamine 2000 (Thermo Scientific, Waltham, MA). At 24-hour post-transfection, cells were harvested in 1×PBS buffer by scraping, centrifuged at 300×g for 5 minutes at 4° C. and stored at −80° C. For infection of adenoviral constructs, LNCaP cells were seeded at 3 to 5×10⁶ cells per 10 cm dish to achieve 80% confluency after 48 hours. Cells were infected at 100 multiplicities of infection (MOI) with either control viral vector or Ad-FLI1 in 3 mL medium with 2% FBS. After 2 hours, 7 mL of RPMI-1640 with 10% FBS were added, and the infection was continued for an additional 48 hours before cells were harvested, pelleted at 4° C. and stored at −80° C.

iv. siRNA Knockdown of ETV-1 Proteins

PC3 cells were seeded at 0.7×10⁶ cells per 10 cm dish in 8 mL of F-12K medium supplemented with 10% FBS one day before transfection. Cells were transfected with a pool of ETV1 specific siRNA duplex oligos (J-003801-06, J-003801-07, J-003801-08 and J-003801-09), or non-targeting (NT) siRNA oligos (cat #0-001810-01-05; Thermo Scientific) at 25 nM and 50 nM concentrations, using Lipofectamine RNAiMax (Thermo Scientific). SiRNA oligos were mixed with Lipofectamine RNAiMAX at 2 nM:1 μL ratio, in 2 mL of OptiMEM I medium, according to manufacturer's instructions. Cells were harvested at 48 hours post-transfection and processed for Western blot analysis.

v. Western Blot Analysis

Proteins were detected in Western blot analysis using ETV4 mouse monoclonal antibody (8D2D2, developed in house), ERG 9FY mouse monoclonal antibody (CM421, Biocare Medical, Pacheco, CA), SPDEF G10 mouse monoclonal antibody (sc-166846, Santa Cruz, CA), FLI1 rabbit monoclonal antibody (D7N5M, 35980S, Cell Signaling Technologies, Danvers, MA), FLAG rabbit polyclonal antibody (F7425, Sigma), and GAPDH rabbit polyclonal antibody (sc-25778, Santa Cruz, CA).

Cell pellets from transfection procedures were lysed in 200 μL M-PER Mammalian Protein Extract Reagent (Thermo Scientific) containing protease and phosphatase inhibitor cocktails (Sigma, St Louis, MO) by sonication at 35% amplitude 15 seconds/15 pulse for 5 minutes in a water bath sonicator. Cell lysates were then cleared by centrifugation at 10,000×g for 15 minutes at 4° C. and transferred to fresh tubes. Protein concentration was measured using Protein Assay Reagent (Bio-Rad, Hercules, CA). Cell lysates equivalent to between two to fifty μg of total protein were separated on 4%-12% NuPAGE bis-tris gel in MES buffer (Thermo Scientific) at 130V for 75 minutes. Separated proteins were transferred onto PVDF membranes using a Trans-Blot SD semi-dry transfer cell (BioRad) at 25 V for 90 minutes. Membranes were blocked in blocking buffer (Tris buffered saline with 0.25% Tween 20 (TBST) containing 1% BSA) for one hour at room temperature before incubation with primary antibodies (1 ng/mL) for 12 hours at 4° C. Membranes were then washed with wash buffer (TBST) in three ten-minute washes before treatment with IRDye 680RD conjugated goat anti-rabbit or IRDye 800 RD conjugated goat anti-mouse secondary antibodies (LI-COR Biosciences, Lincoln, NE) for three hours at room temperature. After incubation, membranes were washed three times with wash buffer. Blots were scanned using the Odyssey® Imaging Systems (LI-COR Biosciences) using the 700 nm channel at medium scan quality, 42 μm resolution, and an intensity setting of 4.

vi. Immunofluorescence Assay

PC3 cells were seeded onto poly-L-lysine coated cover glass (BD Biosciences, San Jose, CA) in F-12K medium supplemented with 10% FBS a day before siRNA transfection. Two days after transfection, cells were fixed with PHEM (60 mM PIPES, 30 mM HEPES, 10 mM EGTA, 8 mM MgSO₄, adjusted to pH 7 with KOH) buffered 4% paraformaldehyde before permeabilization in 1 PHEM with 0.1% Triton X-100. Prior to incubation in primary antibody, cells were blocked in 1% normal horse serum (Vector Laboratories, Burlingame, CA) in PBS. Species-specific secondary antibody, Alexa-Fluor-594 goat anti-rabbit antibody (Thermo Scientific), was subsequently applied, together with 4 μg/mL of DAPI (4′,6-Diamidino-2-Phenylindole). F-actin was stained with Alexa Fluor-488 phalloidin (Thermo Scientific). Images were captured using a 20×/0.45 N S Plan Fluor objective on a Nikon EclipseTs2R inverted microscope equipped with a CoolSNAP DYNO (Photometrics, Tucson, AZ) CCD camera controlled by Nikon NIS Elements (v. 5.11.01) imaging software. Grayscale images were converted into color and merged by using Adobe Photoshop (v.23.2.21).

vii. Peptide Synthesis and Enzyme-Linked Immunosorbent Assay (ELISA)

Peptides were synthesized to achieve ≥70% HPLC purity by a commercial vendor (GenScript Biotech, Piscataway, NJ). Peptides were reconstituted in either water, or with addition of acetonitrile, methanol, 10% acetic acid, or DMSO, depending on the overall charge of each peptide. Key residues required for mAb binding were probed by ELISA using overlapping peptides, truncated peptides and alanine substituted peptides. Reactions were performed in duplicate using NUNC 96-well flatbottom MaxiSorp plates (Thermo Scientific). After coating each well with 500 ng of peptide in 100 μL coating buffer (50 mM NaHCO₃, pH 9.6), the plates were sealed and incubated at 4° C. overnight. The next day, after washing four times with wash buffer (1×PBS+Tween-20, KD Medical, Columbia, MD), the plates were blocked with 200 μL StartingBlock buffer (Thermo Scientific) per well and incubated for 1 hour at room temperature (RT). Plates were then washed once with wash buffer, incubated with 100 μL mAb 29E4 (serially diluted from 1 mg/mL) for 1 hour at 37° C. Plates were further washed four times with wash buffer, and then incubated at 37° C. with 100 μL of a horse radish peroxidase (HRP)-conjugated secondary antibody against the primary antibody, diluted 1:16,000 according to manufacturer's protocols (KPL, Inc., Gaithersburg, MD). After washing four times with wash buffer, 100 μL of K-Blue Aqueous TMB substrate (Neogen, Lexington, KY) was added to the plates and incubated uncovered for 30 minutes at room temperature. To stop the reaction, 100 μL of 2 N Sulphuric acid was added to each well and absorbance were measured immediately at 450 nm. All dilutions of reagents were performed in ELISA diluent (20% NGS in 1×PBS with 0.1% Triton-X 100).

vii. Surface Plasmon Resonance Imaging (SPRi)

Affinity measurements using SPRi was performed using a Biacore T200 instrument (Cytiva, Marlborough, MA) by Creative Biolabs (Shirley, NY, USA). The ETV1 (29E4) antibody was directly immobilized onto a CM5 sensor chip surface using an amine coupling kit (Cytiva). Before immobilization, the CM5 sensor surface was activated using a mixture of 400 mM 1-ethyl-3-(3-dimethylaminopropyl) carbodiimide (EDC) and 100 mM N-hydroxysuccinimide (NHS). The ETV1 antibody was diluted to 40 μg/ml in immobilization buffer (10 mM NaAc, pH 4.5) and injected into Fc4 sample channel at a flow rate of 10 μL/min. The amount of antibody (ligand) immobilized was about 5,000 RU. The chip was deactivated by 1 M Ethanolamine hydrochloride-NaOH (GE Healthcare Life Sciences, Piscataway, NJ) at a flow rate of 10 μL/min for 420 seconds. The reference Fc3 channel underwent similar procedures but without injecting the ligand. The ETV1 immunogenic polypeptide (analyte) was serially diluted with the running buffer to concentrations of 250, 125, 62.5, 31.25, 15.625, 7.813, 3.906, 1.953, and 0 nM, respectively. Different concentrations of the antibody were then injected into the cell over both channels at a flow rate of 30 μL/min, with a contact time of 90 seconds, followed by a dissociation time of 210 seconds. The surface was regenerated with 10 mM Glycine-HCl, pH 1.5 after each injection to prepare for the injection of the next antibody dilution sample. Data analysis was performed on the Biacore T200 computer and with the Biacore T200 evaluation software, using the steady-state affinity model or 1:1 binding model.

ix. Immunohistochemistry and Pathologic Assessment

To evaluate the expression of ETV1 protein in prostate cancer tissue specimens, we screened a tissue microarray (TMA) constructed from prostate tumors of 100 patients who received treatment at the Walter Reed National Military Medical Center. The patients had provided written consent for the collection of biospecimens and clinico-pathologic data under an Institutional Review Board approved protocol (DBS.2020.135). The TMA was made up of tissue cores of 1.0 mm diameter from one to two benign glands, and at least two cores from one or more tumor foci. The cores were arrayed in ten paraffin blocks, each represented by samples from ten patients. Multiple four-micron sections were taken from each block. The first and last samples were stained with H&E to verify each tissue core. The pathological diagnosis of the tissue cores was recorded in the CPDR database.

Preparation and histologic evaluation of formalin fixed paraffin embedded (FFPE) prostate specimens were performed as previously described (Tan et al., Pathology, 2021, 53:205-213). Whole-mounted specimens of cases with ETV1 expression on the TMA were selected for further immunohistochemical examination. Adjacent, four-micron tissue sections were stained with H&E, anti-ERG mouse mAb (9FY, Biocare Medical, Pacheco, CA), and in anti-ETV1 rabbit mAb. For ETV1 staining, sections were dehydrated following deparaffinization, and blocked in 0.6% hydrogen peroxide in methanol for 20 minutes before antigen retrieval in Citrate buffer (pH 6.0) for 15 minutes using a pressure cooker followed by 25 minutes cooling. Sections were then blocked in 10% normal goat serum for 40 minutes before incubation with the primary antibody for 60 minutes at room temperature. The ETV1 mAb was evaluated at 1:40, 1:80, 1:160, and 1:320 dilutions to determine the optimal dilution for protein detection. Sections were then incubated with the biotinylated goat anti-rabbit antibody (Vector Laboratories, Burlingame, CA) for 30 minutes followed by treatment with the ABC Kit (Vector Laboratories) for 30 minutes. Color detection was achieved by treatment with the purple VIP chromogen (Vector Laboratories) for 5 minutes followed by counterstaining in hematoxylin for 1 minutes before slides were dehydrated, cleared, and mounted. For ERG staining, antigen retrieval was performed in EDTA (pH 9.0). After blocking with 1% horse serum for 30 minutes, sections were incubated with the ERG 9FY mAb (Biocare Medical, CA) for 1 hour at room temperature, followed by biotinylated horse anti-mouse antibody at 1:200 dilution for 30 minutes. Color detection was achieved by treatment with VIP chromogen (Vector Laboratories) for 5 minutes followed by counterstaining in hematoxylin. Slides were reviewed by a single genitourinary pathologist (I.A.S.). Cells were recorded as ETV1 positive or when purple VIP chromogen was detected in the nuclei of any tumor cells, and as negative in the absence of any nuclear staining.

### 2. Results

i. Selective Binding of ETV1 Monoclonal Antibody to ETV) Protein

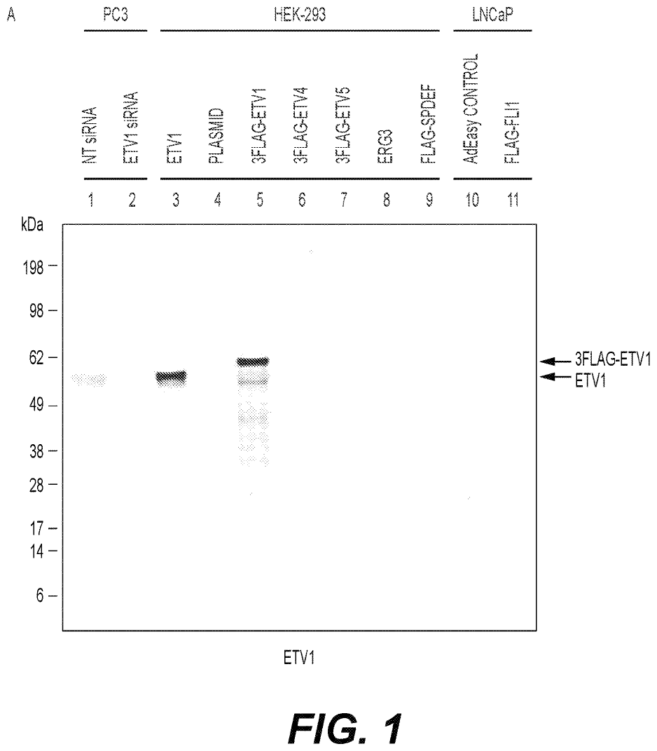

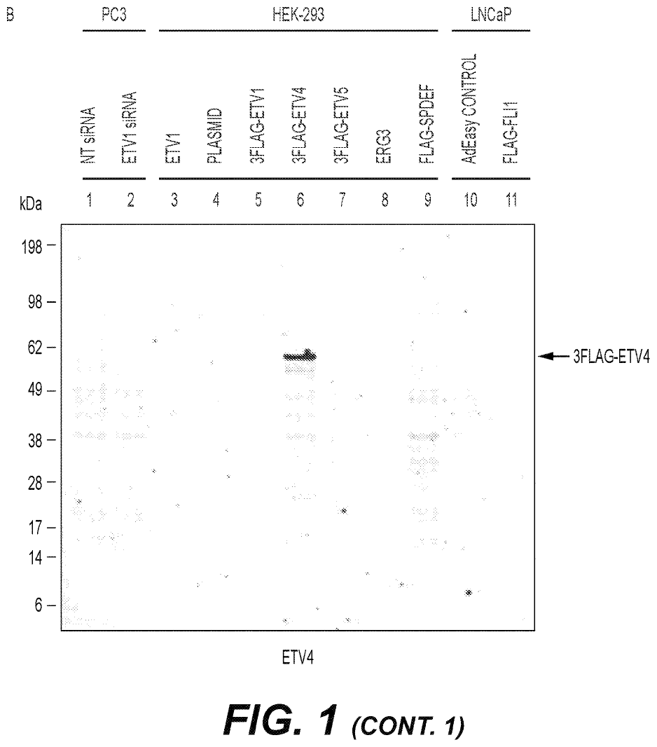

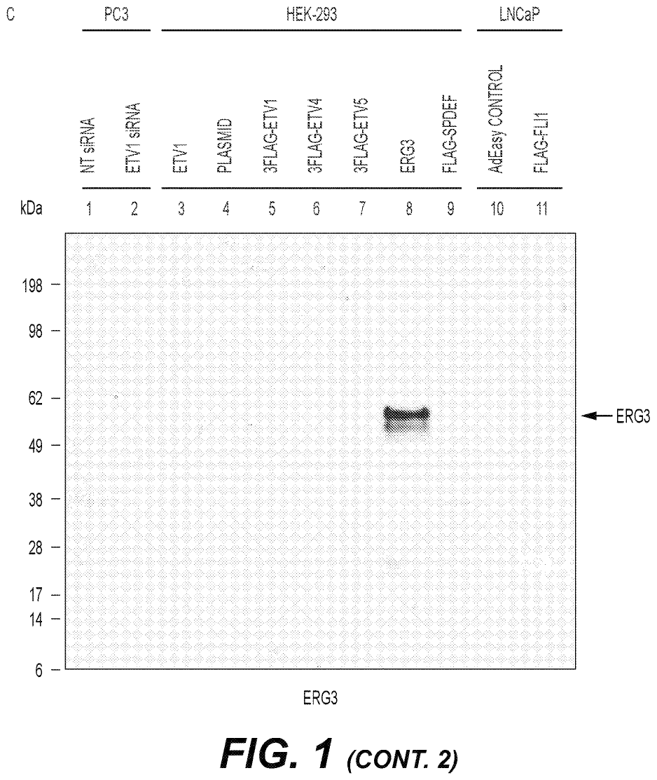

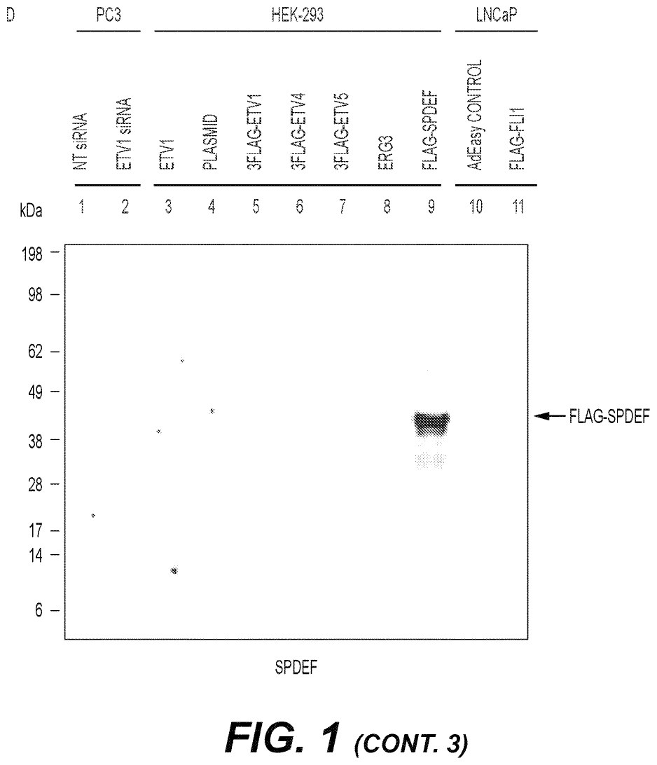

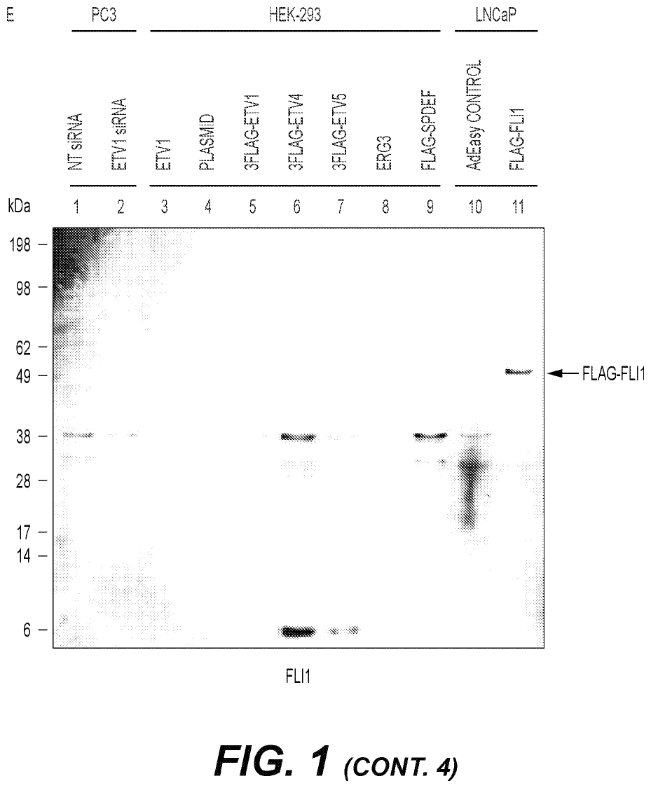

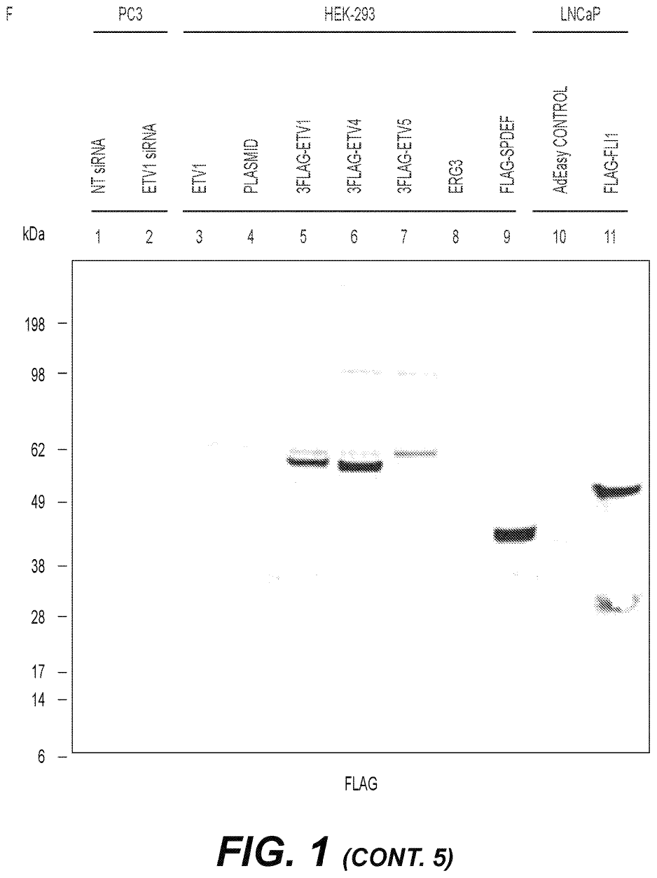

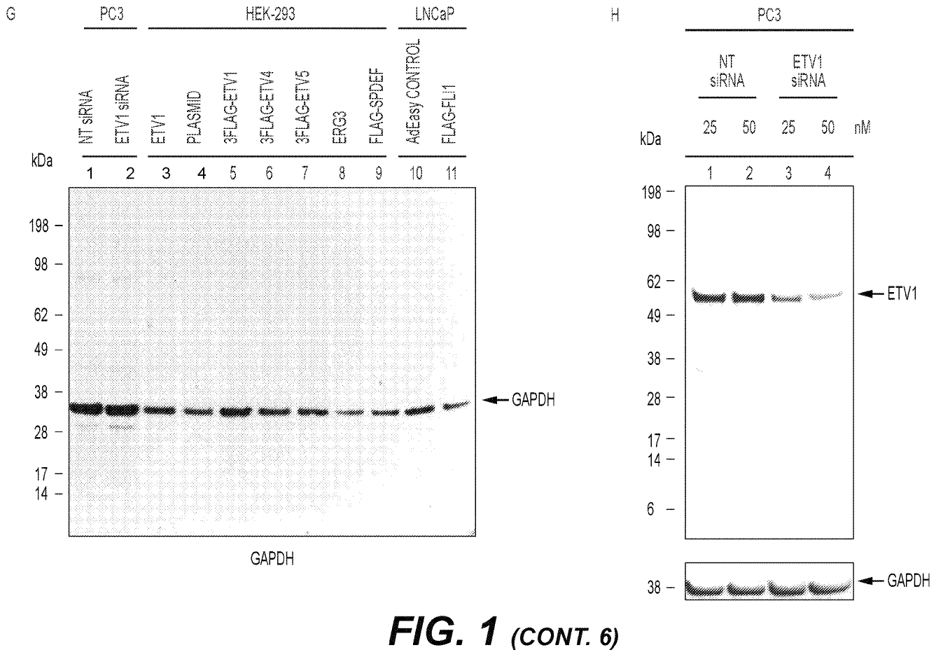

The reactivity of ETV1 rabbit monoclonal antibody against the ETV1 protein was evaluated in immunoblot assays using cell lysates from both endogenously expressed and exogenously expressed ETV1. The antibody stained against a single protein band in PC3 prostate cancer cells and against ETV1 proteins expressed in HEK293 cells (FIG. 1, panel A). The selective binding of the ETV1 mAb is supported by the detection of significant ETV1 protein inhibition following ETV1 siRNA knockdown in PC3 cells (lanes 1-2 in FIG. 1, panel A). More importantly, the ETV1 mAb did not recognize or cross-react with other ETS transcription factors with a high degree of homology to ETV1, including ETV4, ETV5, ERG, SPDEF and FLI1, that were present (FIG. 1, panels B-F). The membrane was re-probed with GAPDH antibody to show loading amounts (FIG. 1, panel G). Since the endogenously expressed ETV1 in PC3 cells was much lower than exogenously expressed protein in HEK-293 cells at the amount of cell lysates loaded in panel A of FIG. 1, an immunoblot of PC3 cell lysates loaded at 50 μg per lane was performed to show the expression of ETV1 in PC3 cells (FIG. 1, panel H). The results show the robust and selective detection of ETV1 by the ETV1 mAb in cell lysates transfected with 25 nM and 50 nM of control siRNA oligonucleotides, which was effectively knocked down by transfection of ETV1 specific oligonucleotides.

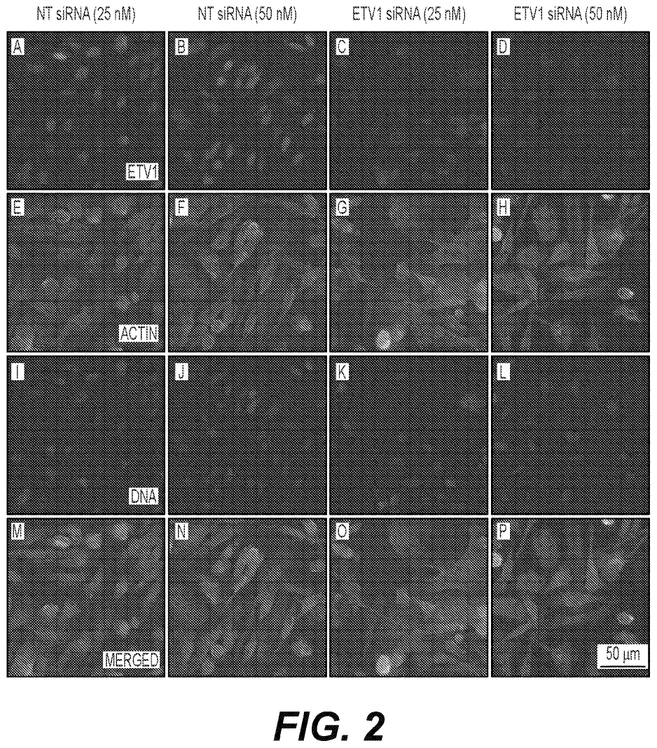

The selective detection of ETV1 protein in PC3 cells by the ETV1 mAb was further validated by immuno-fluorescence staining of PC3 cells. ETV1 was shown to be localized to the nucleus in cells transfected with 25 nM and 50 nM control non-targeting oligonucleotides (FIG. 2, panels A-B). The nuclear localized ETV1 protein detected by the mAb diminished significantly when cells were with transfected the same concentration of ETV1 siRNA oligonucleotides (FIG. 2, panels C-D). Cells were stained with Alexa-488 phalloidin and DAPI to visualize the actin cytoskeleton (FIG. 2, panels E-H) and cell nuclei (FIG. 2, panels I-L), respectively. Merge panels of all three stains are shown in panels M-P of FIG. 2. These results together established the selectivity of the ETV1 mAb in detecting ETV1 protein in prostate cancers cells.

ii. Identification of Minimal Epitope Required for Binding to the ETV1 29E4 mAb

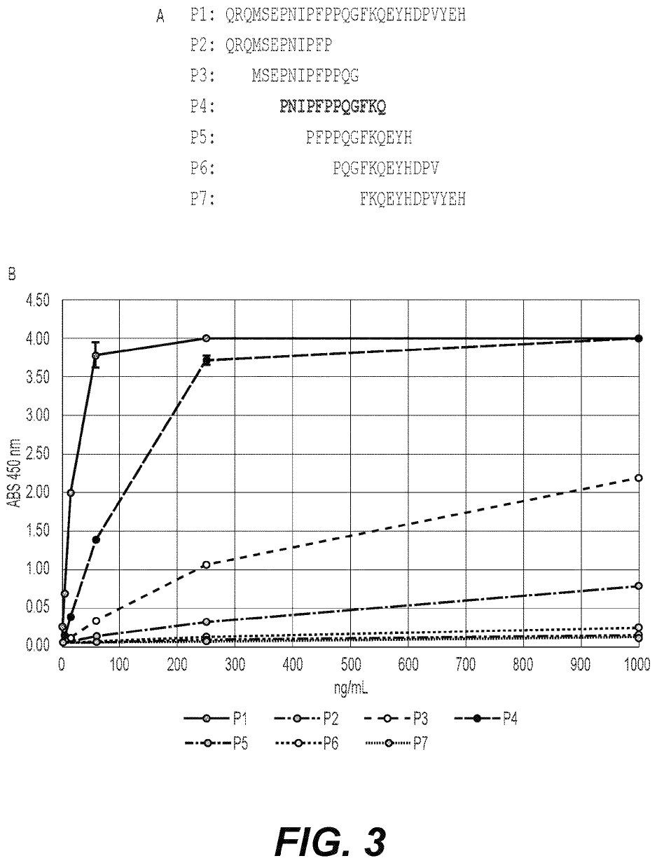

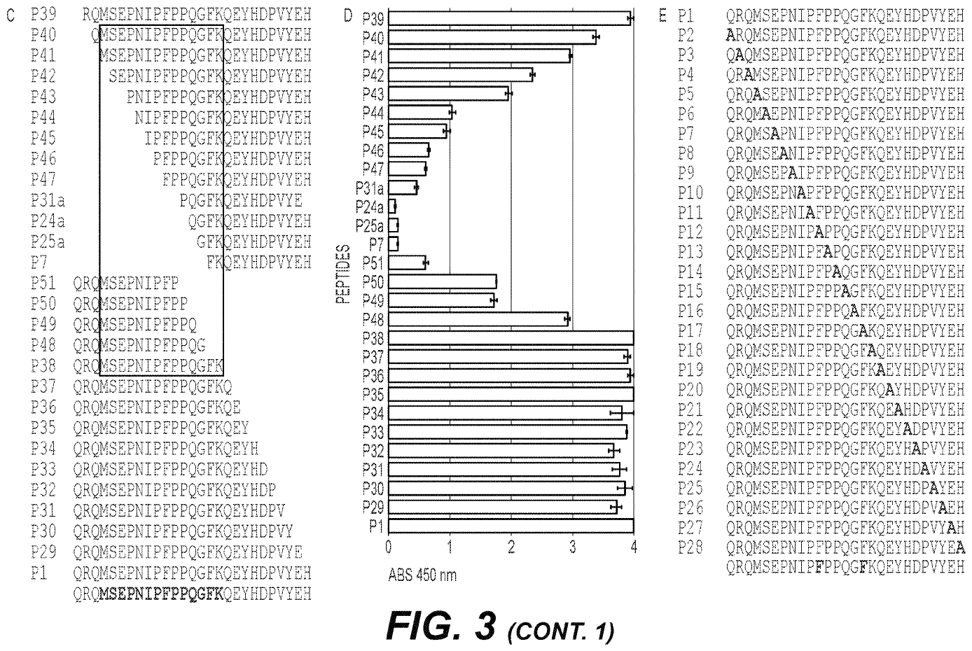

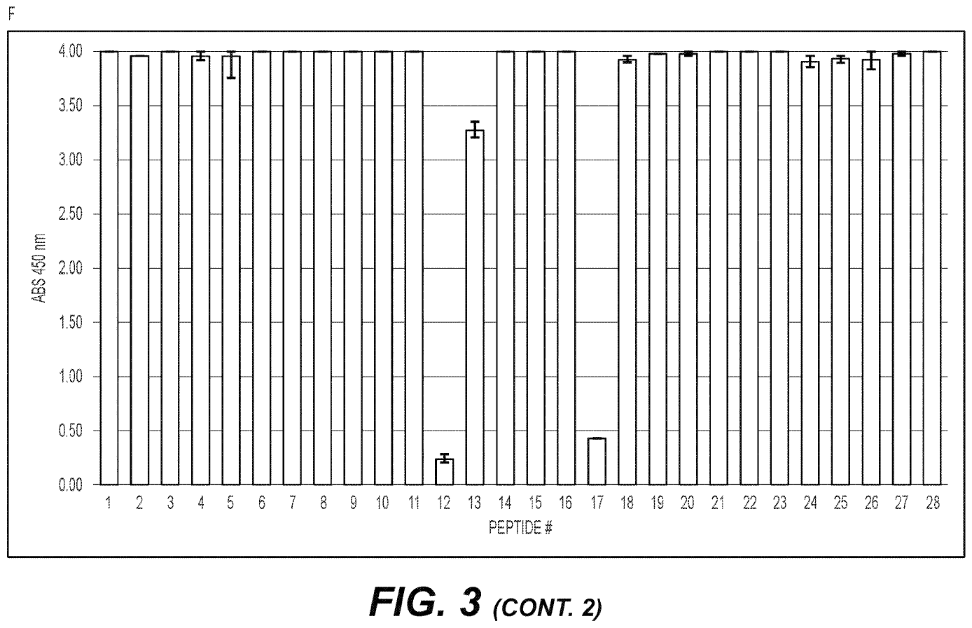

The ETV1 mAb recognizes an epitope within the immunogen peptide, which corresponds to the amino acids 212-238 of the 477 amino acid ETV1 protein (Genbank accession No. P50549; Uniprot accession No. P50549-1). To identify the minimal epitope sufficient for binding of the 29E4 mAb within the immunogenic peptide, a series of six twelve-amino acid peptides (P2-P7) were designed and synthesized with an overlap of nine residues between each consecutive peptide (FIG. 3, panel A). The peptides were evaluated for their ability to bind the ETV 29E4 mAb by ELISA. The 27 amino acid immunogenic peptide, P1, which was used as a positive control, showed the highest reactivity against the antibody. Peptide P4, which comprise residues 218-229, showed the highest reactivity against the antibody, followed by the flanking peptides P3 and P5, respectively. Peptides P2, P6 and P7 showed almost no reactivity against 29E4 (FIG. 3, panel B). These results indicate that the 29E4 mAb minimal recognition sequences are located within a stretch of 18 amino acids, represented by MSEPNIPFPPQGFKQEYH (SEQ ID NO: 18), from residue 215 to 232 of ETV1.

Based on the results from the peptide overlap analysis, a series of peptides with incremental truncations from either the N- or C-terminus of the 27 amino-acid immunogenic peptide were synthesized (FIG. 3, panel C) for evaluated for binding to the ETV1 mAb by ELISA. While peptide P40 (QMSEPNIPFPPQGFKQEYHDPVYEH; SEQ ID NO: 27) and peptide P38 (QRQMSEPNIPFPPQGFK; SEQ ID NO: 42) retained almost 80% to 100% binding efficiency to the 29E4 mAb in comparison to the full-length immunogenic peptide P1, further truncation from the N-terminus and C-terminus, respectively, reduced the binding to about 75% binding efficiency (FIG. 3, panel D). These results suggest that the minimal epitope required for efficient binding to the 29E4 mAb lies within residues 215 to 228 (MSEPNIPFPPQGFK; SEQ ID NO: 16), which is enclosed by the box and highlighted in red in the immunogenic peptide sequence at the bottom of FIG. 3, panel C.

An alanine scanning approach that substituted each amino acid of the immunogenic peptide with an alanine residue was used to determine the epitope at the level of the residue. This method is ideal for evaluating the contribution of side chains of individual amino acids in protein-protein or protein-antibody interaction. The peptides (P1-P28) used to evaluate their binding to the ETV1 mAb are shown in FIG. 3, panel E. The effect of individual alanine substitution on the reactivity of peptides with 29E4 is shown in FIG. 3, panel F. Remarkably, substitution of alanine for residues F222 and F227 (corresponding to peptides P12 and P17, respectively) abolished the binding of these peptides to the antibody almost completely. Thus, these two phenylalanine residues are likely to form the core epitope recognized by the 29E4 mAb. Furthermore, we noted that the adjacent P223 residue positioned downstream to the first core phenylalanine epitope showed a slightly reduced binding in comparison to the wild type P1 and most of the other peptides. This suggests that the proline residue may play an accessory role in the binding and stabilization of the peptide-antibody complex.

iii. Measurement of the Binding Kinetics of the 29E4 Antibody to the Immunogenic Peptides by Surface Plasmon Resonance Imaging

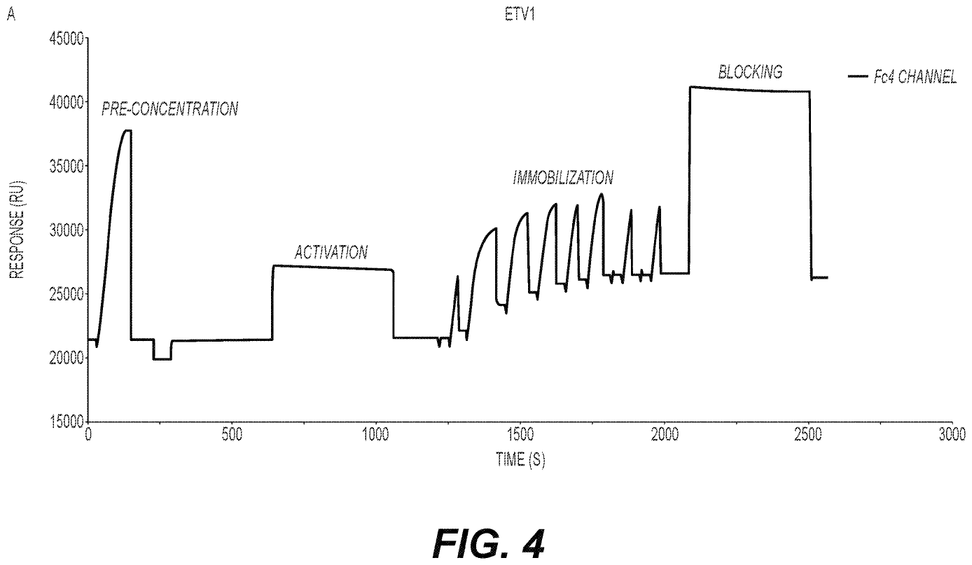

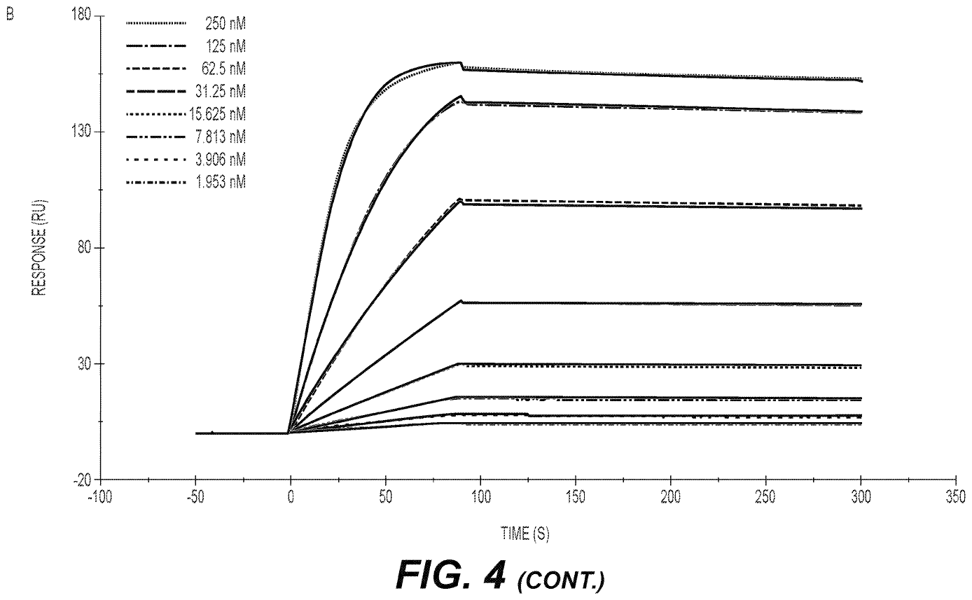

The binding of the 29E4 mAb to its epitope sequence was further analyzed by using SPRi. After immobilizing the ETV1 mAb onto a matrix of dextran on the surface of a Sensor Chip to a level of 5000 response units (RU) with standard amine coupling (FIG. 4, panel A), different concentrations of the serially diluted peptide were then injected as an analyte into the cell over both channels for 300 seconds (contact time of 90 seconds, followed by a dissociation time of 210 seconds) to give a response of about 180 RU. The different binding affinity of each antibody dilution is depicted in the overlay plot of SPR sensorgrams and fitting curves (FIG. 4, panel B). Using the 1:1 binding model, the affinity constant of the ETV1 P1 peptide binding to 29E4 antibody was measured as 479 pM (Table 1).

TABLE 1
Summary of SPRi binding kinetics of the ETV1 mAb

| | |
|---|---|
| Method | CM5 |
| Ligand | ETV1 rabbit monoclonal antibody (29E4) |
| Immobilized Level (RU) | 5023.2 |
| Analyte | Peptide U8498FI280-1 |
| Analyte Conc. | 250-1.953 nM |
| Ka (1/Ms) | 3.11E+05 |
| Kd (1/s) | 1.49E−04 |
| KD (M) | 4.79E−10 |
| Rmax (RU) | 157.15 |
| Chi² (RU²) | 0.84 |
| Fit method | 1:1 binding |

iv. Immunohistochemistry Detection of ETV1 in Prostate Tissue Specimens

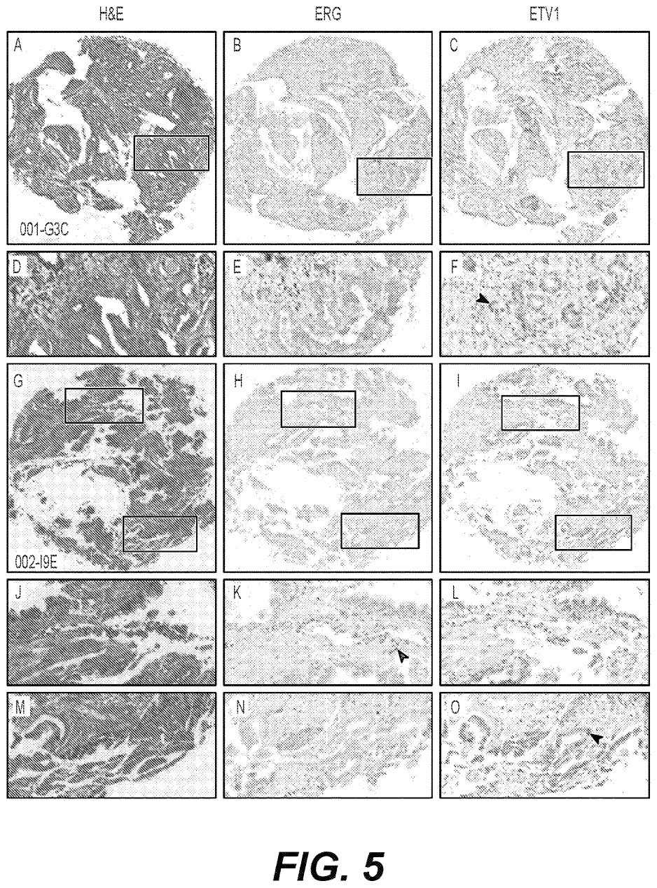

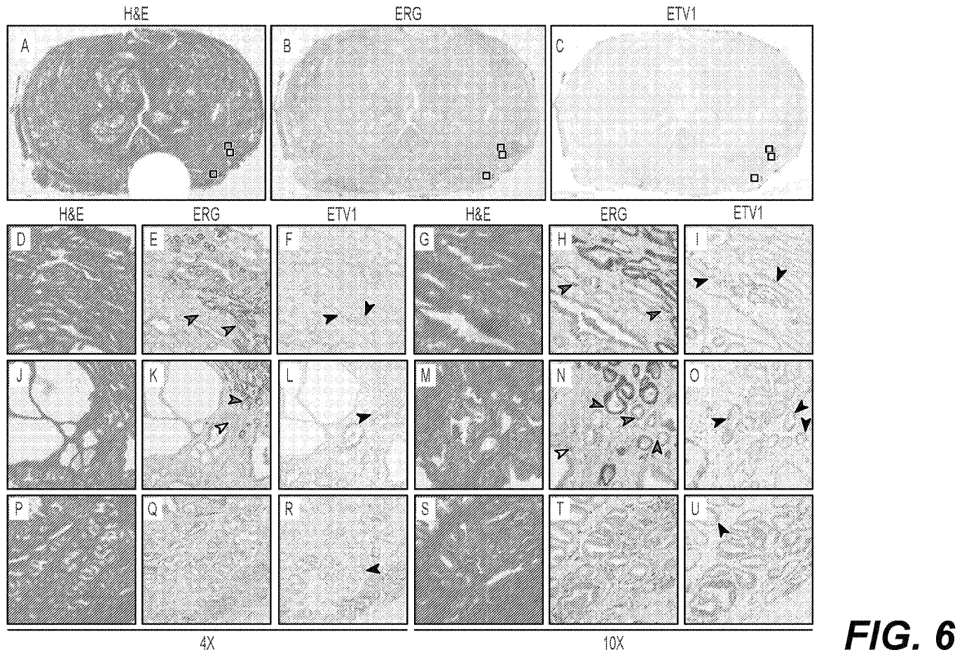

To evaluate the utility of the ETV1 mAb in immunohistochemistry assays, a tissue microarray constructed from 100 cases was probed with the ETV1 mAb. Five cases with positive ETV1 staining were detected, which concurs with the reported range of ETV1 gene fusion frequency in prostate cancer patients. Tissue cores from two of the ETV1 positive cases, 001-G3C and 002-I9E, are highlighted in FIG. 5 (panels A-O). Although it has been reported that ERG and ETV1 gene fusions are mutually exclusive events, we detected a case with both ERG and ETV1 expression (#002-I9E, FIG. 5, panels H-I). Whole mounted sections of this case were further stained with H&E, ERG and ETV1 antibodies (FIG. 6, panels A-D). Morphological examination of the H&E-stained slide shows that ETV1 positive cells appear to have slightly larger nuclei and more copious cytoplasm in comparison to ERG positive cells. While ERG positive prostate tumors show invariable and uniformly positive ERG expression in most cells, as previously reported, ETV1 staining, however, has a less predictable and heterogenous distribution. ETV1 positive (ETV1(+)) tumors typically show glands with a mosaic or skipped staining pattern, which include cells that are partly positive interspersed with ETV1 negative (ETV1(−)) cells (FIG. 6, panels I, O and U). In most of the prostate tumors, ETV1 staining is mutually exclusive from ERG staining. Nonetheless, in the transition area where ETV1 and ERG positive tumors collide, we detected rare hybrid tumor glands that are made up of both ERG positive and ETV1 positive cells (FIG. 6, panels H-I). In these instances, we are unable to ascertain at present if both ETV1 and ERG are expressed in the same cells. Overall, the comparison of the ETV1 mAb-stained whole-mounted prostate section with an ERG mAb-stained adjacent section led us to classify ETV1-stained prostate tumors into four groups: ETV1 negative tumors, ETV1 positive tumors, ETV1 positive tumors with a mosaic or skipping pattern, and hybrid tumors with both ETV1 and ERG positive cells in the same gland.

### 3. Discussion

Oncogenic ETS transcription factors are often overexpressed in various cancers in the presence of other highly homologous tissue specific ETS factors. The ETS transcription factors SPDEF, ELK4, ELF3 and EHF, for example, are highly expressed in the normal prostate epithelium, while, ERG, ETV1, ETV4, ETV5 and FLI1 oncoproteins are overexpressed in prostate cancers only due to genomic rearrangements. Highly selective and specific monoclonal antibodies that distinguish individual ETS oncoproteins from other closely identical ETS factors are essential for their effective use as diagnostic or prognostic reagents.

Mouse monoclonal antibodies have been used routinely for diagnosis and therapy. Proteins that are highly conserved between mice and humans may be recognized as self-antigens in mice. When used as immunogens, tolerance against these proteins often prevents a strong immune response, resulting in the generation of either low affinity or non-specific mAbs or both. The highly homology between the human and mouse ETV1 proteins may have contributed to difficulties in raising a specific ETV1 mAb in mouse. Although increased immune response against highly conserved target molecules could be achieved in animals evolutionarily distant from humans, establishing immortalized antibody-producing cell lines by hybridoma, viral transformation or reprogramming are challenging. Rabbits have been known to produce increased titers of high-affinity antibodies against antigens with conserved epitopes between humans and rodents that may not be immunogenic in mice. Monoclonal antibodies raised in rabbits have also been shown to exhibit higher sensitivity and affinity without loss of antibody specificity. Furthermore, humanized rabbit-derived antibodies have been evaluated for use in antibody-drug-conjugates. These factors prompted us to develop a monoclonal antibody against ETV1 using rabbits as a host animal. The affinity constant of 479 pM exhibited by the ETV1 29E4 mAb is higher than the affinity constant of most mouse monoclonal antibodies and within the affinity constant measured of most rabbit monoclonal antibodies measured using SPRi.

The 27 amino acid immunogen was selected based on several established criteria. Firstly, the peptide is predicted to contain potentially antigenic regions by the EMBOSS antigenic software. Secondly, to avoid possible cross-reactivity, the peptide sequence is confirmed to reside in a region that is non-homologous to other ETS proteins, especially ETV5 and ETV4, which have the highest percent identity to ETV1. Thirdly, since ETV1 is often overexpressed in cancers due to fusion with a 5′ gene partner, amino acids downstream of exon 12 and away from reported gene fusion sites were selected. A multiple sequence alignment of the ETV1 proteins from human, mouse and rabbit showed 97.9% identity between human and mouse ETV1, whereas identity between human and rabbit ETV1 is 95.6%.

Fluorescence in-situ hybridization (FISH) is routinely used to show genomic rearrangements in cancer cells, including ETV1 and other ETS fusions. Although the FISH assay is considered the gold standard for detecting chromosomal translocations or genomic rearrangements, it has some drawbacks. Compared to IHC, the FISH procedure requires longer technologist time to perform, greater interpretation time per case by a pathologist, and substantially higher reagent costs per case examined. A fluorescence microscope is needed to review FISH-treated samples and often a re-examination of the H&E-stained sections is required to confirm areas of invasive carcinoma. IHC interpretation requires only a standard light microscope, and both immunostaining and morphology can be evaluated simultaneously on the same slide. Furthermore, the fluorescent signals of FISH probes are susceptible to quenching and must be stored at −20° C. or lower, while IHC-stained slides can be stored in standard slide folders and the reaction product is permanent.

In multiple cancer types, ETV1 gene fusion also led to elevated mRNA expression, which is confirmed by quantitative RT-PCR, RNA-in situ-hybridization (RISH), or RNA sequencing. The frequent ETV1 expression in GIST and in round cell sarcoma has led to its use as a diagnostic marker detectable by RISH. The use of “Z” linker probes that allow the signal of bound target RNA sequence be amplified in RNAscope has improved the detection of mRNA expression in tumor tissues considerably by overcoming the problem of low transcript levels and instability of RNA molecules. Besides the ability to detect low levels of mRNA transcripts that are intrinsic to the tissue, or a result of tissue degradation commonly observed in clinical FFPE specimens, this approach has very high sensitivity and specificity, and it provides both a quantitative and spatial information of the expressed gene. The major drawback of RNAscope is the significantly higher cost compared to IHC. Discrepancy of results between RNAscope assay and IHC may arise, as gene fusions or frame-shift mutations can truncate the open-reading-frame that render a transcript untranslatable. Hence, RISH is often used together with microarray or IHC to avoid misinterpretations of data or misdiagnoses in clinical samples.

Although higher mRNA expression is accompanied by higher ETV1 protein expression, few studies employ the detection of ETV1 by IHC due to the lack of a specific monoclonal antibody. We expect that the highly specific ETV1 rabbit mAb that we have developed will be a useful reagent for the detection of ETV1 expression in various cancer types and for studying the interaction between ETV1 and other proteins. This antibody may also be more efficacious compared to traditional RISH methods of detection. The molecular characterization of this antibody identified the key residues that bind to ETV1 mAb and confirmed its high affinity. Results from IHC assays validated its utility for detecting ETV1 expression in human prostate tissue. Further evaluation of ETV1 expression in tumor specimens from independent patient cohorts are in progress to establish the sensitivity and specificity of the mAb for diagnosis and stratification of prostate and other cancers.

### Example 2. Structure Characterization of the ETV1 Rabbit Monoclonal Antibody Clone 29E4

The heavy chain variable domain and the light chain variable domain of the ETV1 rabbit monoclonal antibody clone 29E4 described in Example 1 were identified to comprise the amino acid sequence of SEQ ID NO: 1 and 2, respectively: SEQ ID NO: 1 (the heavy chain variable domain of the monoclonal antibody 29E4) QELEESGGDLVKPGASLTLTCTASGFFFSAENHICWVRQAPGKGLEWIGCVYIGSYVGG SGDPYYASWAKGRFTISKTSSTTVTLQMSSLTAADTATYFCARDLGSLWGPGTLVTVSS SEQ ID NO: 2 (the light chain variable domain of the monoclonal antibody 29E4) DVVMTQTASSVSAAVGGTVTIACQSSQSVYDNNNLAWFQQKPGQPPKRLIYSASTLAS GVSSRFKGSGYGTQFTLTISDVQCDDAATYYCVGAFSGYIFVFGGGTEVVVK

Two algorithms were used to identify the Complementary Determining Regions (CDRs) of the heavy and light chains. The CDRs identified using Kabat-Chothia system bioinf.org.uk/abs/info.html) are SEQ ID NO: 3-5 for the heavy chain variable region and SEQ ID NO: 6-8 for the light chain variable region. The CDRs identified using Ofran method (ofranlab.org/paratome/) are SEQ ID NO: 9-11 for the heavy chain variable region and SEQ ID NO: 12-14 for the light chain variable region. The sequences of these CDRs identified are summarized below.

Kabat-Chothia system

| | | | |
|---|---|---|---|
| Heavy chain | CDR1 | GFFFSAENHIC | SEQ ID NO: 3 |
| variable region | CDR2 | CVYIGSYVGGSGDPYY | SEQ ID NO: 4 |
| | CDR3 | DLGSL | SEQ ID NO: 5 |
| Light chain | CDR1 | QSSQSVYDNNNLA | SEQ ID NO: 6 |
| variable region | CDR2 | SASTLAS | SEQ ID NO: 7 |
| | CDR3 | VGAFSGYIFV | SEQ ID NO: 8 |

Ofran method

| | | | |
|---|---|---|---|
| Heavy chain | CDR1 | FFFSAENHIC | SEQ ID NO: 9 |
| variable region | CDR2 | WIGCVYIGSYVGGSGDPYY | SEQ ID NO: 10 |
| | CDR3 | RDLGSL | SEQ ID NO: 11 |
| Light chain | CDR1 | QSVYDNNNLA | SEQ ID NO: 12 |
| variable region | CDR2 | RLIYSASTLAS | SEQ ID NO: 13 |
| | CDR3 | VGAFSGYIFV | SEQ ID NO: 14 |

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all constructs, methods, and/or component features, steps, elements, or other aspects thereof can be used in various combinations.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or the entire group members are present in, employed in, or otherwise relevant to a given product or process. Furthermore, it is to be understood that the disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. In general, where embodiments or aspects of the disclosure, is/are referred to as comprising particular elements, features, etc., certain embodiments or aspects consist, or consist essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification.

All patents, patent applications, websites, other publications or documents, accession numbers and the like cited herein are incorporated by reference in their entirety for all purposes to the same extent as if each individual item were specifically and individually indicated to be so incorporated by reference.

What is claimed is:

1. A monoclonal antibody, or an antigen-binding fragment thereof, that binds to an epitope of ETV1 (ETS Variant Transcription Factor 1), comprising a heavy chain variable domain and a light chain variable domain, wherein:
a) the heavy chain variable domain comprises a complementarity determining region 1 (CDR1) comprising the amino acid sequence of SEQ ID NO: 3, a CDR2 comprising the amino acid sequence of SEQ ID NO: 4, and a CDR3 comprising the amino acid sequence of SEQ ID NO: 5, and wherein the light chain variable domain comprises a CDR1 comprising the amino acid sequence of SEQ ID NO: 6, a CDR2 comprising the amino acid sequence of SEQ ID NO: 7, and a CDR3 comprising the amino acid sequence of SEQ ID NO: 8; or
b) the heavy chain variable domain comprises a CDR1 comprising the amino acid sequence of SEQ ID NO: 9, a CDR2 comprising the amino acid sequence of SEQ ID NO: 10, and a CDR3 comprising the amino acid sequence of SEQ ID NO: 11, and wherein the light chain variable domain comprises a CDR1 comprising the amino acid sequence of SEQ ID NO: 12, a CDR2 comprising the amino acid sequence of SEQ ID NO: 13, and a CDR3 comprising the amino acid sequence of SEQ ID NO: 14.

2. The monoclonal antibody, or the antigen-binding fragment thereof, of claim 1, wherein the heavy chain variable domain comprises an amino acid sequence having at least 90% sequence identity to SEQ ID NO: 1 and the light chain variable domain comprises an amino acid sequence having at least 90% sequence identity to SEQ ID NO: 2.

3. The monoclonal antibody, or the antigen-binding fragment thereof, of claim 1 or 2, wherein the heavy chain variable domain comprises the amino acid sequence of SEQ ID NO: 1 and the light chain variable domain comprises the amino acid sequence of SEQ ID NO: 2.

4. The monoclonal antibody, or the antigen-binding fragment thereof, of any one of claims 1-3, wherein the monoclonal antibody is a rabbit monoclonal antibody.

5. The monoclonal antibody, or the antigen-binding fragment thereof, of any one of claims 1-4, wherein the epitope of ETV1 comprises the amino acid sequence of SEQ ID NO: 16.

6. The monoclonal antibody, or the antigen-binding fragment thereof, of any one of claims 1-5, wherein the epitope of ETV1 comprises the amino acid sequence of SEQ ID NO: 15.

7. A pharmaceutical composition comprising the monoclonal antibody, or the antigen-binding fragment thereof, of any one of claims 1-6, and a pharmaceutically acceptable excipient.

8. A nucleic acid molecule encoding the monoclonal antibody, or the antigen-binding fragment thereof, of any one of claims 1-6.

9. A host cell comprising the nucleic acid molecule of claim 8.

10. A method for determining the likelihood of a subject having a cancer, the method comprising:
a) determining a level of ETV1 expression in a sample obtained from the subject using the monoclonal antibody, or the antigen-binding fragment thereof, of any one of claims 1-6; and
b) comparing the level of ETV1 expression obtained in step a) with a reference level of ETV1 expression,
wherein the reference level of ETV1 expression is obtained from a subject known not to have a cancer and an increased level of ETV1 expression in the sample as compared to the reference level of ETV1 expression indicates that there is an increased likelihood that the subject has a cancer, or wherein the reference level of ETV1 expression is obtained from a subject known to have a cancer and a decreased level of ETV1 expression in the sample as compared to the reference level of ETV1 expression indicates that there is an increased likelihood that the subject does not have a cancer.

11. The method of claim 10, wherein the level of ETV1 expression in the sample is determined using an analysis selected from the group consisting of Western blotting, protein gel electrophoresis, immunoprecipitation, ELISA, immunohistochemistry, and combinations thereof.

12. The method of claim 10 or 11, wherein the level of ETV1 expression in the sample is determined by performing immunohistochemistry on the sample.

13. The method of any one of claims 10-12, wherein the subject is a human suspected of having a cancer.

14. The method of any one of claims 10-13, wherein the cancer is prostate cancer, gastrointestinal stromal tumor, small round cell sarcoma, melanoma, or gliomas.

15. A method of diagnosing a cancer associated with overexpression of ETV1 in a subject in need thereof, the method comprising:
a) determining a level of ETV1 expression in a sample obtained from the subject using the monoclonal antibody, or the antigen-binding fragment thereof, of any one of claims 1-6; and
b) comparing the level of ETV1 expression obtained in step a) with a reference level of ETV1 expression,
wherein the reference level of ETV1 expression is obtained from a subject known not to have a cancer and an increased level of ETV1 expression in the sample as compared to the reference level of ETV1 expression indicates that there is an increased likelihood that the subject has a cancer, or wherein the reference level of ETV1 expression is obtained from a subject known to have a cancer associated with overexpression of ETV1 and a decreased level of ETV1 expression in the sample as compared to the reference level of ETV1 expression indicates that there is an increased likelihood that the subject does not have a cancer.

16. The method of claim 15, wherein the level of ETV1 expression in the sample is determined using an analysis selected from the group consisting of Western blotting, protein gel electrophoresis, immunoprecipitation, ELISA, immunohistochemistry, and combinations thereof.

17. The method of claim 15 or 16, wherein the level of ETV1 expression in the sample is determined by performing immunohistochemistry on the sample.

18. The method of any one of claims 15-17, wherein the subject is a human suspected of having a cancer associated with overexpression of ETV1.

19. The method of any one of claims 15-18, wherein the cancer associated with overexpression of ETV1 is prostate cancer, gastrointestinal stromal tumor, small round cell sarcoma, melanoma, or gliomas.

20. A method of treating a cancer associated with overexpression of ETV1, the method comprising administering to a subject in need thereof a therapeutically effective amount of the monoclonal antibody, or the antigen-binding fragment thereof, of any one of claims 1-6, or the pharmaceutical composition of claim 7.

21. The method of claim 20, wherein the cancer associated with overexpression of ETV1 is prostate cancer, gastrointestinal stromal tumor, small round cell sarcoma, melanoma, or gliomas.

22. A method of detecting ETV1 expression in a sample, the method comprising incubating the sample with the monoclonal antibody, or the antigen-binding fragment thereof, of any one of claims 1-6 under conditions permitting the detection of ETV1 expression when ETV1 is present in the sample.

23. The method of claim 22, wherein the ETV1 expression is detected using an analysis selected from the group consisting of Western blotting, protein gel electrophoresis, immunoprecipitation, ELISA, immunohistochemistry, and combinations thereof.

24. The method of claim 22 or 23, wherein the sample is a biological sample comprising cells.

25. An immunogenic composition comprising an epitope of ETV1, wherein the epitope of ETV1 comprises the amino acid sequence of SEQ ID NO: 15 or SEQ ID NO: 16.

26. The epitope of claim 25, wherein the epitope comprises the amino acid of SEQ ID NO: 16 and contains no more than 30 amino acids, or wherein the epitope comprises the amino acid sequence of SEQ ID NO: 15 and contains no more than 15 amino acids.

27. The immunogenic composition of claim 25 or 26, further comprising an adjuvant.

28. A method of generating a monoclonal antibody that binds to ETV1, the method comprising administering the immunogenic composition of any one of claims 25-27 to a non-human mammal, isolating B cells from the non-human mammal, immortalizing the B cells to create a cell line capable of producing a monoclonal antibody, and selecting the monoclonal antibody that binds to the epitope of ETV1 comprising the amino acid sequence of SEQ ID NO: 15 or SEQ ID NO: 16.

29. A kit for use in detecting ETV1 expression, the kit comprising:
a) the monoclonal antibody, or the antigen-binding fragment thereof, of any one of claims 1-6, in a container;
b) a control; and
c) instructions for carrying out an immunoassay for determination of ETV1 expression.

30. The kit of claim 29, wherein the control is a control slide comprising a tissue sample known to have ETV1 expression.
